(19) United States
(12) Patent Application Publication
(10) Pub. No.: US 2008/0146562 A1
(43) Pub. Date: Jun. 19, 2008

(54) HALOGENATED QUINAZOLINYL NITROFURANS AS ANTIBACTERIAL AGENTS

(75) Inventors: Suzanne Chamberland, Eastman (CA); Francois Malouin, Eastman (CA); Roger Leger, Saint-Lambert (CA); Ving J. Lee, Los Altos, CA (US)

Correspondence Address: KLARQUIST SPARKMAN, LLP, 121 SW SALMON STREET, SUITE 1600, PORTLAND, OR, 97204, US

(73) Assignee: Ulysses Pharmaceutical Products Inc.,

(21) Appl. No.: 11/871,897

(22) Filed: Oct. 12, 2007

Related U.S. Application Data
(63) Continuation-in-part of application No. 10/567,660, filed on Feb. 8, 2006.
(60) Provisional application No. 60/493,336, filed on Aug. 8, 2003.

(51) Int. Cl. A61K 31/5377; C07D 239/72; A61K 31/517; A61P 31/04; A01N 43/54; A01N 43/60; A01N 43/84; A01P 1/00; A61K 31/496; C07D 295/00

(52) U.S. Cl. 514/234.5; 544/293; 514/266.24; 514/252.17; 544/116

(57) ABSTRACT

The present invention includes novel compounds of the formula

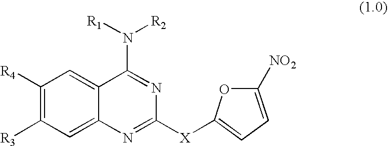

wherein: X is absent, or trans or cis CHCH; R₁ is (C₁-C₁₀)alkyl unsubstituted or substituted by one to three hydroxy, (C₂-C₁₀)alkenyl unsubstituted or substituted by one to three hydroxy, (C₂-C₁₀)alkynyl unsubstituted or substituted by one to three hydroxy, or aryl unsubstituted or substituted by one to three hydroxy; R₂ is hydrogen, alkyl or aryl, wherein R₁ and R₂ when taken together form a (C₂-C₁₀)cycloalkyl unsubstituted or substituted by one to three hydroxy; and R₃ and R₄ are, independently of each other, H, halogen, or

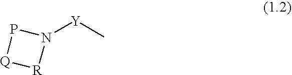

wherein: P and R are each independently selected from CH₂, CH₂CH₂ and CH₂CHT, wherein T is alkyl; Q is O, S, NH or NCH₃; and Y is absent or —CH₂CH₂)ₙO—, and n=1 or 2; with the proviso that at least one of R₃ and R₄ is halogen, and that when R₄ is halogen, R₃ is hydrogen and Y is absent, neither R₁ nor R₂ are alkyl; or pharmaceutically acceptable salts thereof.

The invention also includes pharmaceutically acceptable formulations of said compounds which exhibit antibiotic activity against a wide spectrum of microorganisms including organisms which are resistant to multiple antibiotic families and are useful as antibacterial agents for treatment or prophylaxis of bacterial infections, or their use as antiseptics, agents for sterilization or disinfection.

### CROSS-REFERENCE TO RELATED APPLICATIONS

This application is a continuation-in-part of U.S. patent application Ser. No. 10/567,660, which is the National phase of PCT international application PCT/CA2004/001466, which has the international filing date of Aug. 6, 2004, and entered U.S. National phase on Feb. 8, 2006, which U.S. patent application claims the benefit of U.S. Provisional Patent Application No. 60/493,336 filed Aug. 8, 2003, which are incorporated herein by reference.

### FIELD OF THE INVENTION

This invention relates to novel nitrofuran antibiotics and their use for the treatment or prophylaxis of bacterial infections in humans or animals, or their use as antiseptics, sterilizants or disinfectants. These compounds exhibit antibiotic activity against a wide spectrum of microorganisms, including organisms which are resistant to multiple antibiotic families.

### BACKGROUND OF THE INVENTION

The following review of the background of the invention is merely provided to aid in the understanding of the present invention and neither it nor any of the references cited within it are admitted to be prior art to the present invention.

Management of nosocomial or community-acquired bacterial infections is becoming very difficult due to the emergence of bacteria resistant to one or multiple families of antibiotics. Unfortunately, the widespread and indiscriminant use of antibiotics has led to a rapid increase in the number of bacterial strains which are resistant to antibiotics. Most importantly, resistance has emerged among clinically important microorganisms which threaten the utility of the currently available arsenal of antibiotics. A global trend of increasing resistance to antibiotics, with wide variations according to geographical areas, is well documented by the World Health Organization and in the scientific literature.

There is a need for novel and effective antibiotics that are particularly active against microorganisms which are resistant to currently available drugs. For example, resistance of bacteria causing urinary tract infections to trimethoprim-sulfamethoxazole, β-lactams and fluoroquinolones is becoming a major factor in the management of such infections. Despite the use of nitrofuran antibiotics for several decades, mainly for the treatment of urinary tract infections, resistance to agents of this family has remained low (0-2%) in microorganisms most commonly encountered (Gupta K. Addressing antibiotic resistance. Dis Mon. 2003 February; 49(2):99-110; Nicolle L E. Urinary Tract Infection: Traditional pharmacologic therapies. 2003. February; 49(2):111-128).

U.S. Pat. Nos. 3,970,648, 3,973,021 and 3,974,277 disclose nitrofurans of the following formulae: 2-[2-(5-nitro-2-furyl)vinyl]-4-(anilino)quinazoline, 2-[2-(5-nitro-2-furyl)vinyl]-4-(p-hydroxyanilino)-quinazoline, 2-[2-(5-nitro-2-furyl)vinyl]-4-(o-hydroxyanilino)-quinazoline, and 2-[2-(5-nitro-2-furyl)vinyl]-4-(m-hydroxyanilino)-quinazoline. These patents teach the use of these compounds as pesticides and animal growth promotants for improving feed efficiency in animals such as poultry, swine and cattle. Although these molecules gained the property of being adequate edible feed additives for animal growth promotion compared to quinazoline molecules having the nitrofuran group directly attached to it (U.S. Pat. No. 3,542,784), a drawback of the compounds from the above patents (U.S. Pat. Nos. 3,970,648, 3,973,021 and 3,974,277) is that the patents teach that they are now devoid of activity against important pathogens such as Escherichia coli, Staphylococcus aureus and Salmonella. It would be desirable to obtain nitrofurans which provide significant improvement of potency and expand the antimicrobial spectrum of activity. This means that lower amounts of compounds are required for in vitro and in vivo (in animals) antimicrobial action against a wider variety of pathogens affecting animals and humans.

Besides, there are only a few nitrofuran antibiotics currently used in humans for the treatment of infectious diseases and one is known by the generic name nitrofurantoin (commercial names include: Macrobid, Macrodantin, Furadantin). It is used in adults and children to treat acute urinary tract infections and to prevent recurrent urinary tract infections. A drawback of nitrofurantoin is that it does not have good potency (i.e., relatively high amounts are required to exert its antibacterial activity) and it does not have a wide spectrum of antimicrobial activity, which limits the use of this compound in treating bacterial infections.

Novel nitrofurans with superior antimicrobial potency and improved pharmacological properties would provide an alternative for the treatment of severe infections caused by antibiotic-susceptible and -resistant microorganisms.

### SUMMARY OF THE INVENTION

The compounds described herein can be used as antibiotics for the treatment or prophylaxis of bacterial infections, or as antiseptics, sterilizants, or disinfectants.

The general structural feature of the compounds is a nitrofuran linked to the 2 position of a quinazoline directly or via a vinyl group. It is believed that the nitrofuran is essential for antimicrobial activity while the quinazoline in particular as substituted, e.g., with an halogen and/or a methylpiperazino group, has at least one of the following effect: improves potency, expands the spectrum of activity (e.g., activity against E. coli, S. aureus, Salmonella, Mycobacterium, anaerobic bacteria and microorganisms that are resistant to multiple antibiotics), provides a bactericidal (lethal) activity (i.e., as opposed to a bacteriostatic growth-inhibitory activity), provides in vivo activity, and/or improves solubility.

The quinazoline contains one or two functional groups at the 4 position attached via an amine, and a hydrogen, halogen, or solubilizing group (such as an amine containing heterocyclic group, or more preferably an amine containing heterocyclic group which further contains at least one oxygen or nitrogen group) at the 6 or 7 position with the proviso that at least one of the 6 or 7 positions are substituted with a halogen.

This invention includes compounds of the following general formula:

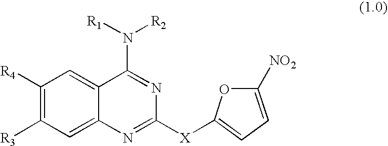

wherein

X is absent, or trans or cis CHCH;

R₁ is (C₁-C₁₀)alkyl unsubstituted or substituted by one to three hydroxy, (C₂-C₁₀)alkenyl unsubstituted or substituted by one to three hydroxy, (C₂-C₁₀)alkynyl unsubstituted or substituted by one to three hydroxy, or aryl unsubstituted or substituted by one to three hydroxy;

R₂ is hydrogen, alkyl, or aryl,

wherein R₁ and R₂ when taken together form a (C₂-C₁₀)cycloalkyl unsubstituted or substituted by one to three hydroxy; and

R₃ and R₄ are, independently of each other, H, halogen, or

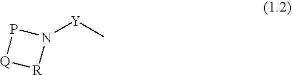

wherein:

P and R are each independently selected from CH₂, CH₂CH₂ and CH₂CHT, wherein T is alkyl;

Q is O, S, NH or NCH₃; and

Y is absent or —(CH₂CH₂O)ₙ—, and n=1 or 2;

with the proviso that at least one of R₃ and R₄ is halogen, and that when R₄ is halogen, R₃ is hydrogen and Y is absent, neither R₁ nor R₂ are alkyl;
or pharmaceutically acceptable salts thereof.

The invention also includes pharmaceutically acceptable formulations of said compounds which exhibit antibiotic activity against a wide spectrum of microorganisms including organisms which are resistant to multiple antibiotic families and are useful as antibacterial agents for treatment or prophylaxis of bacterial infections, or their use as antiseptics, agents for sterilization or disinfection. In another aspect of the present invention there is provided compositions comprising the compounds of the invention. In yet another aspect of the present invention there is provided processes for preparing the compounds of the invention. Certain terms that are used in this application are defined below.

The term “alkyl” refers to the radical of saturated aliphatic groups including straight chain alkyl groups, branched-chain alkyl groups, cycloalkyl (alicyclic) groups, alkyl substituted cycloalkyl groups, and cycloalkyl substituted alkyl groups. Typical alkyl groups include, but are not limited to, methyl, ethyl, propyl, isopropyl, butyl, isobutyl, t-butyl, pentyl, isopentyl, hexyl, etc. The alkyl group is preferably (C₁-C₁₀) alkyl, and more preferably (C₁-C₆) alkyl and even more preferably (C₂-C₄) alkyl.

The term “alkyl” can encompass heteroalkyl groups wherein one or more carbons of the hydrocarbon backbone are replaced with a heteroatom, e.g. N, O or S. The term “alkyl” can encompass a “substituted alkyl” having substituents replacing a hydrogen on one or more carbons of the hydrocarbon backbone. Such substituents can include, for example, halogen, hydroxyl, carbonyl (such as carboxyl, ketones (including alkylcarbonyl and arylcarbonyl groups), and esters (including alkyloxycarbonyl and aryloxycarbonyl groups)), thiocarbonyl, acyloxy, alkoxyl, phosphoryl, phosphonate, phosphinate, amino, acylamino, amido, amidine, imino, cyano, nitro, azido, sulfhydryl, alkylthio, sulfate, sulfonate, sulfamoyl, sulfonamido, heterocyclyl, aralkyl, or an aromatic or heteroaromatic moiety. The moieties substituted on the hydrocarbon chain can themselves be substituted, if appropriate. For instance, the substituents of a substituted alkyl may include substituted and unsubstituted forms of aminos, azidos, iminos, amidos, phosphoryls (including phosphonates and phosphinates), sulfonyls (including sulfates, sulfonamidos, sulfamoyls and sulfonates), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like. Any substituted alkyl may have 1 to 5 substituents or any combinations of 1 to 5 substituents.

The terms “alkenyl” and “alkynyl” refer to unsaturated aliphatic groups analogous in length and possible substitution to the alkyls described above, preferably (C₂-C₁₀), and more preferably (C₂-C₆) alkyl and even more preferably (C₂-C₄), but that contain at least one double or triple bond respectively. An “alkenyl” is an unsaturated branched, straight chain, or cyclic hydrocarbon radical with at least one carbon-carbon double bond. The radical can be in either the cis or trans conformation about the double bond(s). Typical alkenyl groups include, but are not limited to, ethenyl, propenyl, isopropenyl, butenyl, isobutenyl, tert-butenyl, pentenyl, hexenyl, etc. An “alkynyl” is an unsaturated branched, straight chain, or cyclic hydrocarbon radical with at least one carbon-carbon triple bond. Typical alkynyl groups include, but are not limited to, ethynyl, propynyl, butynyl, isobutynyl, pentynyl, hexynyl, etc.

The term “aryl” refers to aromatic radicals having 3-14 ring atoms and at least one ring having a conjugated pi electron system. Preferably at least two, more preferably at least four, of the ring atoms are carbon atoms. For example aryl may be a C₅, C₆, C₇, C₈, C₉ or C₁₀ ring. The term “aryl” encompasses “heteroaryl” compounds. The term “heteroaryl” refers to an aromatic heterocyclic group usually with one or more heteroatoms selected from O, S and N in the ring. Examples of aryl include without limitation phenyl, substituted phenyl, pyridyl, substituted pyridyl, pyridinyl, substituted pyridinyl, thiophenyl, substituted thiophenyl, furanyl, substituted furanyl, thiazole, oxazole or substituted or unsubstituted imidazole. Such substituents can include, for example, halogen, hydroxyl, carbonyl (such as carboxyl, ketones (including alkylcarbonyl and arylcarbonyl groups), and esters (including alkyloxycarbonyl and aryloxycarbonyl groups)), thiocarbonyl, acyloxy, alkoxyl, phosphoryl, phosphonate, phosphinate, amino, acylamino, amido, amidine, imino, cyano, nitro, azido, sulfhydryl, alkylthio, sulfate, sulfonate, sulfamoyl, sulfonamido, heterocyclyl, aralkyl, or an aromatic or heteroaromatic moiety. The moieties substituted on the hydrocarbon chain can themselves be substituted, if appropriate. For instance, the substituents of a substituted aryl may include substituted and unsubstituted forms of aminos, azidos, iminos, amidos, phosphoryls (including phosphonates and phosphinates), sulfonyls (including sulfates, sulfonamidos, sulfamoyls and sulfonates), and silyl groups, as well as ethers, alkylthios, carbonyls (including ketones, aldehydes, carboxylates, and esters), —CF₃, —CN and the like. Such substituted aryl may have 1 to 5 substituents or any combinations of 1 to 5 substituents.

The term “halogen” refers to fluoro, chloro, bromo or iodo, or fluoride, chloride, bromide or iodide, or fluorine, chlorine, bromine or iodine.

The present invention includes the pharmaceutically acceptable salts of the compounds defined by general formula 1.0.

The term “pharmaceutically acceptable salt” as used herein, refers to salts of the compounds of the invention which are substantially nontoxic to living organisms e.g. sulfate, pyrosulfate, bisulfate, sulfite, bisulfite, phosphate, monohydrogenphosphate, dihydrogenphosphate, metaphosphate, pyrophosphate, bromide, hydrobromide, iodide, acetate, propionate, decanoate, caprate, caprylate, acrylate, ascorbate, formate, hydrochloride, dihydrochloride, isobutyrate, caproate, heptanoate, propiolate, glucuronate, glutamate, propionate, phenylpropionate, salicylate, oxalate, malonate, succinate, suberate, sebacate, fumarate, malate, maleate, hydroxymateate, mandelate, mesylate, nicotinate, isonicotinate, cinnamate, hippurate, nitrate, stearate, phthalate, teraphthalate, butyne-1,4-dioate, butyne-1,4-dicarboxylate, hexyne-1,4-dicarboxylate, hexyne-1,6-dioate, benzoate, chlorobenzoate, methylbenzoate, hydrozybenzoate, methoxybenzoate, dinitrobenzoate, o-acetoxybenzoate, naphthalene-2-benzoate, phthalate, p-toluenesulfonate, p-bromobenzenesulfonate, p-chlorobenzenesulfonate, xylenesulfonate, phenylacetate, trifluoroacetate, phenylpropionate, phenylbutyrate, citrate, lactate, alpha-hydroxybutyrate, glycolate, tartrate, hemitartrate, benzenesulfonate, methanesulfonate, ethanesulfonate, propanesulfonate, hydroxyethanesulfonate, 1-naphthalenesulfonate, 2-naphthalenesulfonate, 1,5-naphthalenedisulfonate, mandelate, tartarate, and the like.

### DESCRIPTION OF THE FIGURES

FIG. 1 is a time-kill curve for Example I (Compound V), Example VII (Compound XV), and ciprofloxacin against S. aureus ATCC 29213.

FIG. 2 is a time-kill curve for Example I (Compound V) and ciprofloxacin against E. coli ATCC 25922.

FIG. 3 illustrates in vivo activity of compound Example I in a S. aureus model of systemic infection in the mouse.

### DETAILED DESCRIPTION

Compounds of the present invention of formula I generally contain a nitrofuran linked to a quinazoline ring directly or by a vinyl group. The quinazoline ring contains one or two functional groups at the 4 position attached via an amine, a halogen at the 6 position or 7 position or both and at either the 6 position or 7 position, a hydrogen, a halogen or solubilizing group (such as an amine containing heterocycle or more preferably a heterocyclic containing at least one nitrogen and an oxygen or nitrogen group), and a nitrofuran moiety attached to the 2 position directly or by a vinyl group.

In an embodiment, there is provided a compound of formula I, wherein when either R₁ or R₂ is H, then the other is not an aromatic ring substituted with a carboxylic acid.

In a more particular embodiment, there is provided a compound of formula I, wherein when either R₁ or R₂ is H, then the other is not an aromatic ring substituted with a carboxylic acid attached on the ring at position 3.

In another embodiment, there is provided a compound of formula I, wherein when either R₁ or R₂ is an aromatic ring, then the other is not an alkyl or an acetyl.

Compounds of the present invention can generally be made using the following methods. To 5-fluoro-anthranilamide hydrochloride is added, in steps, hydrochloric acid, acetic anhydride and aqueous ammonia, forming 6-fluoro-2-methyl-4-(3H)quinazolinone. Next 5-nitro-2-furancarboxaldehyde is added with acetic anhydride and sulfuric acid to form 6-fluoro-2-[2-(5-nitro-2-furyl)vinyl]-4-(3H)quinazolinone (III), which is used to prepare chloro and anilino derivatives. For example, phosphorus pentachloride and phosphorus oxychloride were added to form 6-fluoro-2-[2-(5-nitro-2-furyl)vinyl]-4-chloroquinazoline (IV) to which various functional groups can be added to the 4 position on the quinazoline. We refer to the Examples for a more detailed description of these methods.

### Antimicrobial Data

Overview. In vitro and in vivo (in animals) tests have revealed the unique antimicrobial properties of 7-fluoro-2-[2-(5-nitro-2-furyl)vinyl]-4-(p-hydroxyanilino)quinazoline and derivatives, and demonstrated that the spectrum of activity of these molecules is highly suitable for treatment of difficult-to-treat human infections. In particular, 7-fluoro-2-[2-(5-nitro-2-furyl)vinyl]-4-(p-hydroxyanilino)quinazoline and 6-(morpholino)-7-fluoro-2-[2-(5-nitro-2-furyl)vinyl]-4-(p-hydroxyanilino)-quinazoline are highly potent broad-spectrum antibacterial agents that demonstrated activity against multiple Gram positive, Gram negative bacteria (TABLE 2). Such a property is comparable, or better, to extremely potent commercial drugs of the macrolide, β-lactam, or fluoroquinolone class. Moreover, the nitrofurans of the present invention like 7-fluoro-2-[2-(5-nitro-2-furyl)vinyl]-4-(p-hydroxyanilino)-quinazoline and 6-(morpholino)-7-fluoro-2-[2-(5-nitro-2-furyl)vinyl]-4-(p-hydroxyanilino)-quinazoline, being of a different structural class, are not affected by commonly found microbial mechanisms of resistance that have been developed over the recent years against most antimicrobial agents currently used clinically. Also, we were able to demonstrate that (E)-2,2′-(6-fluoro-7-(4-methylpiperazin-1-yl)-2-(2-(5-nitrofuran-2-yl)vinyl)quinazolin-4-ylazanediyl)diethanol, administrated by gavages (TABLE 3), is active in vivo in a mouse model of infection, thus indicating oral bioavailability and relatively low toxicity. All these antimicrobial and chemical properties represent those of a potent and safe antibiotic molecule.

In various embodiments, the nitrofurans of the present invention may be used therapeutically in formulations or medicaments to prevent or treat bacterial infections. The invention provides corresponding methods of medical treatment, in which a therapeutic dose of a nitrofuran of the present invention is administered in a pharmacologically acceptable formulation, e.g. to a patient or subject in need thereof. Accordingly, the invention also provides therapeutic compositions comprising a nitrofuran of the present invention, and a pharmacologically acceptable diluent, adjuvant, excipient or carrier. In one embodiment, such compositions include a nitrofuran of the present invention in a therapeutically or prophylactically effective amount sufficient to treat or prevent a bacterial infection. The therapeutic composition may be soluble in an aqueous solution at a physiologically acceptable pH.

A “therapeutically effective amount” refers to an amount effective, at dosages and for periods of time necessary, to achieve the desired therapeutic result, such as a reduction of bacterial infection. A therapeutically effective amount of a nitrofuran of the present invention may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as preventing or inhibiting the rate of bacterial infection-related disease onset or progression. A prophylactically effective amount can be determined as described above for the therapeutically effective amount. For any particular subject, specific dosage regimens may be adjusted over time according to the individual need and the professional judgment of the person administering or supervising the administration of the compositions.

As used herein “pharmaceutically acceptable carrier” or “excipient” includes any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, a nitrofuran of the present invention can be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolid acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating the active compound (e.g. a nitrofuran of the present invention) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. In accordance with an alternative aspect of the invention, a nitrofuran of the present invention may be formulated with one or more additional compounds that enhance the solubility of the nitrofuran.

In accordance with another aspect of the invention, therapeutic compositions of the present invention, comprising a nitrofuran of the present invention, may be provided in containers or commercial packages which further comprise instructions for use of the nitrofuran for the prevention and/or treatment of bacterial infection.

Accordingly, the invention further provides a commercial package comprising a nitrofuran of the present invention, or the above-mentioned therapeutic composition, together with instructions for the prevention and/or treatment of bacterial infection.

The invention further provides a use of a nitrofuran of the present invention for prevention and/or treatment of bacterial infection. The invention further provides a use of a nitrofuran of the present invention for the preparation of a medicament for prevention and/or treatment of bacterial infection.

The invention further provides a use of a nitrofuran of the present invention as an antiseptic, sterilizant, or disinfectant.

Now in order to more particularly define some embodiments of the present invention, the following Examples provide details of specific compounds of the invention, methods of producing the same and results from testing such compounds, and these Examples are not to be construed as limiting the scope of the invention.

### EXAMPLE I

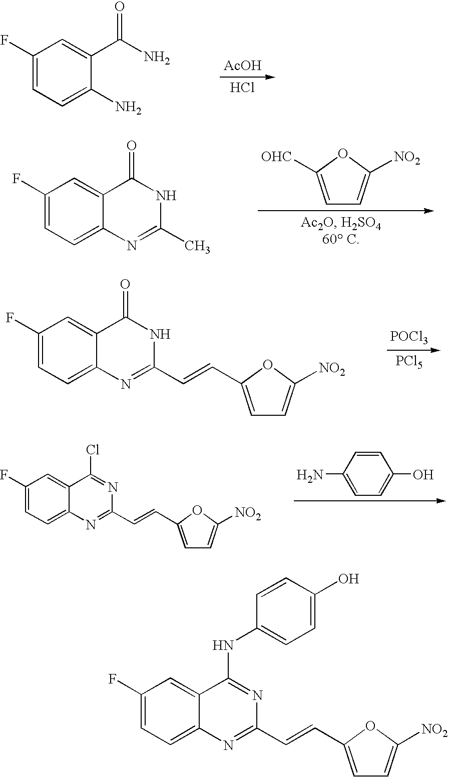

6-Fluoro-2-methyl-4-(3H) quinazolinone (I):

5-Fluoro-anthranilamide hydrochloride was prepared by adding 20 ml of concentrated hydrochloric acid (37% by weight) to a solution of 27.3 g of 5-fluoro-anthranilamide in 200 ml of methanol. This mixture was cooled in an ice bath to precipitate the hydrochloride which was then collected and dried to obtain a product. A 17.4 g (0.1 mole) portion of the hydrochloride thus obtained was refluxed for 3 hours with 100 ml acetic anhydride and allowed to stand overnight. The mixture was then cooled in an ice bath and the solids collected by filtration on a Buchner funnel. The filter cake was slurried in 100 ml of water, and warmed to enhance dissolution and then 28% aqueous ammonia was added until the mixture was alkaline. After cooling, the 6-fluoro-2-methyl-4-(3H)quinazolinone precipitated as a solid, was then collected, washed with a small amount of cold water and dried at 70° C. to obtain the desired product.

5-nitro-2-furancarboxaldehyde (II): A total of 86.5 g of 5-nitrofurfurylidine diacetate was added in small portions to 90 ml of sulfuric acid (73% by weight) over a period of 10 to 15 min. The mixture was stirred for 30 min at ambient temperature, 10 min at 50° C., cooled to 30° C., and then poured onto 150 g of crushed ice. The mixture was filtered, sucked as dry as possible on a Buchner funnel with the aid of a rubber dental dam and this afforded 51.5 g of 5-nitro-2-furancarbox-aldehyde which melted at 32°-34° C.

6-fluoro-2-[2-(5-nitro-2-furyl)vinyl]-4-(3H)quinazolinone (III): To 16 g (0.1 mole) 6-fluoro-2-methyl-4-(3H)quinazolinone were added 100 ml acetic anhydride, 0.5 ml 96% sulfuric acid and 20 g (0.14 mole) 5-nitro-2-furancarboxaldehyde and the mixture was stirred 2 hours at 50°-60° C. The reaction mixture was poured into water and boiled 10 min. After it stood overnight, the product was collected by filtration, washed with water, then methanol. A yellow solid was obtained. This solid 6-fluoro-2-[2-(5-nitro-2-furyl)vinyl]-4-(3H)quinazolinone was used to prepare the chloro-(IV) and anilino (V) derivatives described below.

6-fluoro-2-[2-(5-nitro-2-furyl)vinyl]-4-chloroquinazoline (IV): A 500 ml 3 necked flask fitted with a stirrer, reflux condenser and protected by a calcium chloride trap was charged with 9.0 g of phosphorus pentachloride (0.043 mole) and 70 ml of phosphorus oxychloride and the mixture stirred. To this 11.3 g (0.04 mole) of 6-fluoro-2-[2-(5-nitro-2-furyl)vinyl]-4-(3H)quinazolinone was added and rinsed into the flask with 15 ml of phosphorus oxychloride. The mixture was heated under reflux for 4 hours, cooled in an ice bath and diluted with 150 ml of diethyl ether. The 6-fluoro-2-[2-(5-nitro-2-furyl)vinyl]-4-chloroquinazoline which precipitated was collected by filtration, washed with 100-150 ml of diethyl ether, slurried in 100 ml of diethyl ether and then refiltered to obtain 8.09 g of the desired product.

6-fluoro-2-[2-(5-nitro-2-furyl)vinyl]-4-(p-hydroxyanilino)-quinazoline (V): A 250 ml Erlenmeyer flask equipped with a magnetic stirrer and oil bath for heating was charged with 8.0 g (0.07 mole) of p-aminophenol and 25 ml of dimethylformamide. After the p-aminophenol was dissolved by stirring, (0.03 mole) of 6-fluoro-2-[2-(5-nitro-2-furyl)vinyl]-4-chloroquinazoline (IV) was added. The reaction mixture was then heated at 70° C.-90° C. for 2 hours after which 60 ml of water was added and the solution after cooling was placed in a refrigerator for crystallization. After 3 days, the brown yellow solid was collected, washed first with water, then methanol and then dried to obtain 7.20 g of product.

### EXAMPLE II

6-fluoro-2-[2-(5-nitro-2-furyl)vinyl]-4-(m-hydroxyanilino)-quinazoline (VI): An Erlenmeyer flask is charged with 4.8 g (0.044 mole) of m-aminophenol and 100 ml of dimethylformamide. The charge is stirred to dissolve the m-aminophenol and 6.5 g (0.02 mole) of 6-fluoro-2-[2-(5-nitro-2-furyl)vinyl]-4-chloroquinazoline (IV) is added. The reaction mixture is reacted as in Example I to obtain 6.5 g of crude product, a yellow solid which melts at 241°-242° C. with decomposition. A 5.5 g sample is recrystallized from 40 ml of dimethyl formamide and 74 ml of methanol is added to the warm solution which is then cooled to recrystallize the purified product.

### EXAMPLE III

6-fluoro-2-[2-(5-nitro-furyl)vinyl]-4-(o-hydroxyanilino)-quinazoline (VII): An Erlenmeyer flask equipped with magnetic stirrer and oil bath for heating is charged with 5.0 g (0.046 mole) of o-aminophenol and 100 ml of dimethylformamide. The charge is stirred to dissolve o-aminophenol and 6.0 g (0.02 mole) of 6-fluoro-2-[2-(5-nitro-2-furyl)vinyl]-4-chloroquinazoline (IV) added. The reaction mixture is reacted at 80° to 90° C. for 2 hours to form an organic precipitate; 100 ml of water is added to the warm mixture which is then allowed to cool and placed overnight in a refrigerator to crystallize. The solids are collected, washed with methanol and dried to obtain 7.5 g of brown-tan solid. A solution of the product in 100 ml of dimethylformamide is treated with activated carbon and filtered. A first portion of 75 ml of methanol is added to the warm filtrate then an additional 25 ml portion. Cooling and scratching gives 5.5 g of orange crystals of the purified product.

### EXAMPLE IV

6-fluoro-2-[2-(5-nitro-2-furyl)vinyl]-4-anilinoquinazoline (VIII): A 250 ml flask equipped with stirrer, reflux condenser and thermometer is charged with 4.1 g (0.044 mole) aniline and 100 ml dimethyl formamide. The charge is stirred to dissolve and 6 g (0.02 mole) 6-fluoro-2-[2-(5-nitro-2-furyl)vinyl]-4-chloroquinazoline (IV) is added. The mixture is reacted at 130°-132° C. for 2 hours to form a dark red solution. A 75 ml portion of water is added to the warm solution which is allowed to stand at room temperature overnight, then cooled 1 hour in an ice bath. The crystallized solid is collected, washed with methanol and dried to yield 5.5 g of brown solid. The solid is dissolved in 50 ml warm dimethyl formamide, decolorized with activated carbon, and precipitated by adding 100 ml methanol, with cooling and scratching to induce crystallization. The precipitated solid is collected and washed with methanol to yield the desired product.

### EXAMPLE V

7-fluoro-2-[2-(5-nitro-2-furyl)vinyl]-4-(p-hydroxyanilino)-quinazoline (IX): This compound is prepared in the same manner as 6-fluoro-2-[2-(5-nitro-2-furyl)vinyl]-4-(p-hydroxyanilino)-quinazoline (example I) but starting with 4-fluoroanthranilamine (27 g).

The synthesis of similar (non-nitrofuran) 6-halogenated quinazoline compounds is described in the following references.

Synthesis and histamine H2-antagonist activity of 4-quinazolinone derivatives. Ogawa, Nobuo; Yoshida, Toshihiko; Aratani, Takayuki; Koshinaka, Eiichi; Kato, Hideo; Ito, Yasuo. Chemical & Pharmaceutical Bulletin (1988), 36(8), 2955-67.

Synthesis and biological evaluation of 2-styrylquinazolin-4(3H)-ones, a new class of antimitotic anticancer agents which inhibit tubulin polymerization. Jiang, Jack B.; Hesson, D. P.; Dusak, B. A.; Dexter, D. L.; Kang, G. J.; Hamel, E. Journal of Medicinal Chemistry (1990), 33(6), 1721-8.

Kuo, Sheng-chu; Hour, Mann-jen; Huang, Li-jiau; Lee, Kuo-hsiung. Preparation of 2-phenyl-4-quinazolinones and 2-phenyl-4-alkoxy-quinazolines as anticancer and antiplatelet drugs. U.S. (2002), 23 pp.

6-Alkylamino- and 2,3-Dihydro-3′-methoxy-2-phenyl-4-quinazolinones and Related Compounds: Their Synthesis, Cytotoxicity, and Inhibition of Tubulin Polymerization. Hour, Mann-Jen; Huang, Li-Jiau; Kuo, Sheng-Chu; Xia, Yi; Bastow, Kenneth; Nakanishi, Yuka; Hamel, Ernest; Lee, Kuo-Hsiung. Journal of Medicinal Chemistry (2000), 43(23), 4479-4487.

### EXAMPLE VI

6,7-Difluoro-2-Methyl-4-(3H)quinazolinone (X): 4,5-fluoro-Anthranilamide hydrochloride was prepared by adding 10 ml of concentrated hydrochloric acid (37% by weight) to a solution of 10 g of 4,5-difluoroanthranilamide in 100 ml of methanol.

This mixture was cooled in an ice bath to precipitate the hydrochloride which was then collected and dried to obtain a product. A (0.01 mole) portion of the hydrochloride thus obtained was refluxed for 3 hours with 10 ml acetic anhydride and allowed to stand overnight. The mixture was then cooled in an ice bath and the solids collected by filtration on a Buchner funnel. The filter cake was slurried in 10 ml of water, and warmed to enhance dissolution and then 28% aqueous ammonia was added until the mixture was alkaline. After cooling the 6,7-difluoro-2-methyl-4-(3H)quinazolinone precipitated as a solid, was then collected, washed with a small amount of cold water and dried at 70° C. to obtain the desired product.

6,7-Difluoro-2-[2-(5-nitro-2-furyl)vinyl]-4-(p-hydroxyanilino)quinazoline (XI): This compound was prepared in the same manner as that described in Example I or the synthesis of 6-fluoro-2-[2-(5-nitro-2-furyl)vinyl]-4-(p-hydroxyanilino)quinazoline, by using 6,7-difluoro-2-methyl-4-(3H)quinazolinone (1 g) as a starting material.

The synthesis of 4,5-fluoro-anthranilamide is described in the following references.

Hayes, Thomas K.; Kiely, John S. Tricyclic tetrahydroquinoline derivatives and tricyclic tetrahydroquinoline combinatorial libraries. PCT Int. Appl. (1998), 119 pp. WO 9834111 A1 19980806.

Hayes, Thomas K.; Forood, Behrouz; Kiely, John S. 4-Substituted quinoline derivatives and 4-substituted quinoline combinatorial libraries. PCT Int. Appl. (1998), 124 pp. WO 9834115 A1 19980806.

Gao, Yun. Compositions containing N-amino- and N-hydroxy-quinazolinones and methods for preparing combinatorial libraries thereof. .S. (2001), 15 pp. U.S. Pat. No. 6,184,377 B120010206.

Desos, Patrice; Lepagnol, Jean M.; Morain, Philippe; Lestage, Piere; Cordi, Alex A. Structure-Activity Relationships in a Series of 2[1H]-Quinolones Bearing Different Acidic Function in the 3-Position: 6,7 Dichloro-2-[1H]-oxoquinoline-3-phosphonic Acid, a New Potent and Selective AMPA/Kainate Antagonist with Neuroprotective Properties. Journal of Medicinal Chemistry (1996), 39(1), 197-206.

Sadhu, Chanchal; Dick, Ken; Treiberg, Jennifer; Sowell, C. Gregory; Kesicki, Edward A.; Oliver, Amy. Preparation of Purinylquinazolinones as Inhibitors of Human phosphatidylinositol 3-kinase delta. U.S. Pat. Appl. Publ. (2002), 86 pp., Cont.-in-part of U.S. Ser. No. 841,341. US 2002161014 A1 20021031.

Sadhu, Chanchal; Dick, Ken; Treiberg, Jennifer; Sowell, C. Gregory; Kesicki, Edward A.; Oliver, Amy. Quinazolinone derivatives as inhibitors of human phosphatidylinositol 3-kinase delta. PCT Int. Appl. (2001), 278 pp WO 0181346 A2 20011101.

### EXAMPLE VII

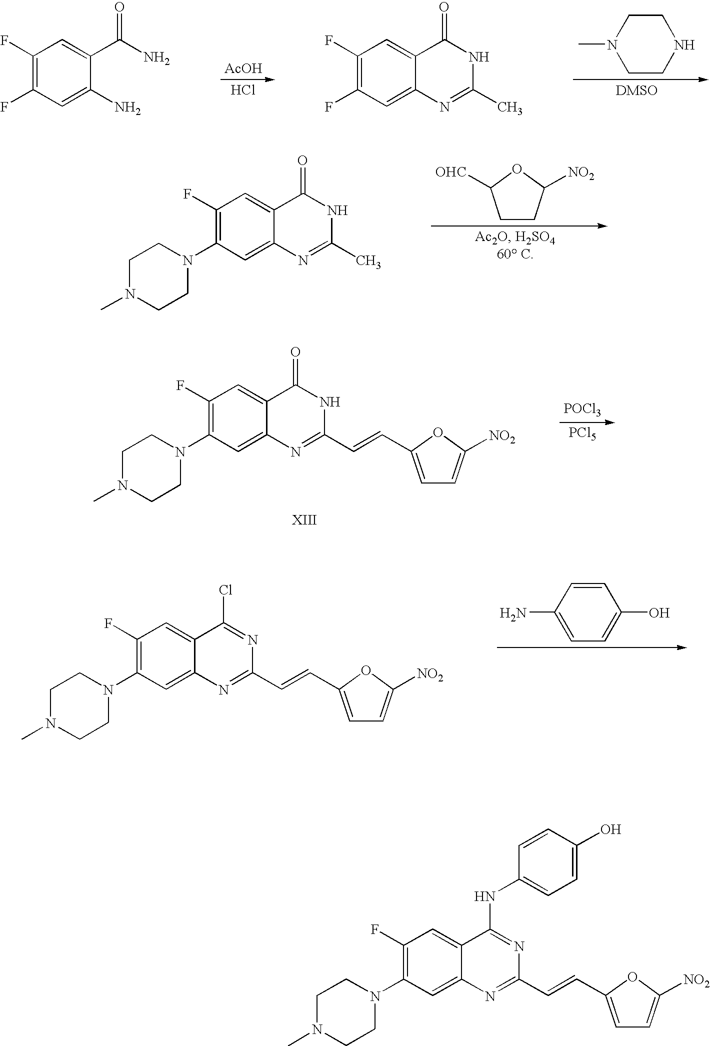

7-(4-methylpiperazino)-6-fluoro-2-Methyl-4-(3H)quinazolinone (XII): To a solution of 6,7-Difluoro-2-Methyl-4-(3H)quinazolinone (X) (5 mmol) in DMSO (10 ml) was added 4-methyl-piperidine (20 mmol). The mixture was heated to 80° C. for 4 h. After cooling water (30 ml) was added and the solid was collected by filtration. The product was further purified by flash chromatography.

7-(4-methylpiperazino)-6-fluoro-2-[2-(5-nitro-2-furyl)vinyl]-4-(3H)quinazolinone (XIII): This compound was prepared in the same manner as described for 6-fluoro-2-[2-(5-nitro-2-furyl)vinyl]-4-(3H)quinazolinone (III). Thus the desired product was obtained starting with XII (3 mmol).

7-(4-methyl piperazino)-6-fluoro-2-[2-(5-nitro-2-furyl)vinyl]-4-chloroquinazoline (XIV): This compound was prepared in the same manner as described for 6-fluoro-2-[2-(5-nitro-2-furyl)vinyl]-4-chloroquinazoline (IV). Thus the desired product was obtained starting with XIII (1.5 mmol).

7-(4-methylpiperazino)-6-fluoro-2-[2-(5-nitro-2-furyl)vinyl]-4-(p-hydroxyanilino)-quinazoline (XV): This compound was prepared in the same manner as described for 6-fluoro-2-[2-(5-nitro-2-furyl)vinyl]-4-(p-hydroxyanilino)quinazoline (V). Thus the desired product was obtained starting with XIII (1.5 mmol).

### EXAMPLE VIII

### Compounds 1 to 3

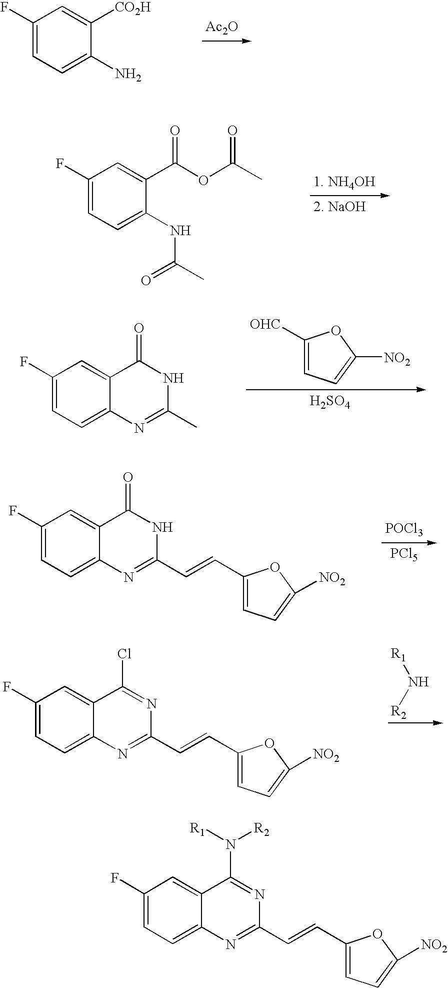

6-Fluoro-2-methyl-3H-quinazolin-4-one: A solution of 2-amino-5-fluorobenzoic acid (23.9 g) in acetic anhydride (125 ml) was heated to reflux for 2 h. The acetic anhydride was removed in vacuo. The residue was added to conc. Ammonium hydroxide (500 ml) and stirred 1 h at ambient temperature. Sodium hydroxide 10% w/v (100 ml) was added and the resulting mixture was concentrated in vacuo to half the volume. The solution was adjusted to pH 2 using conc. HCl and the resulting precipitate was filtered, washed with water and dried by azeotrope with toluene (3×300 ml) to afford the title compound (30.8 g) as a white solid.

6-Fluoro-2-[2-(5-nitro-furan-2-yl)-vinyl]-3H-quinazolin-4-one: A mixture of 6-Fluoro-2-methyl-3H-quinazolin-4-one (25 g), 5-nitro-2-furaldehyde (25 g) and concentrated sulfuric acid (0.25 ml) in acetic acid was heated at 120° C. for 2 days. The reaction mixture was cooled, filtered and the solid washed with ethyl acetate to afford the title compound (26.2 g) as a greening yellow solid.

4-Chloro-6-fluoro-2-[2-(5-nitrofuran-2-yl)-vinyl]quinazoline: A solution of 6-Fluoro-2-[2-(5-nitro-furan-2-yl)-vinyl]-3H-quinazolin-4-one (26.2 g), phosphorous pentachloride (39 g) and phosphorus oxychloride (250 ml) was heated to reflux for 2 days. The reaction mixture was cooled and diluted with ether (500 ml), filtered and the solid was washed with ether. The title compound (20 g) was obtained upon drying as a yellow solid.

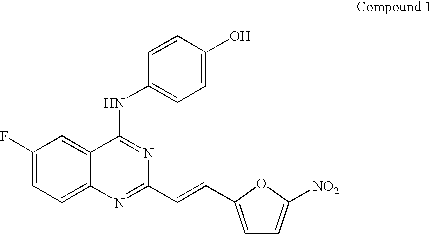

(E)-4-(6-fluoro-2-(2-(5-nitrofuran-2-yl)vinyl)quinazolin-4-ylamino)phenol: A mixture of 4-Chloro-6-fluoro-2-[2-(5-nitrofuran-2-yl)-vinyl]quinazoline (1 g) and 4-aminophenol (0.85 g) in dimethylformamide (45 ml) was heated to 90° C. for 2 h. The reaction mixture was cooled, poured into water (100 ml) and filtered. The solid was washed with water (50 ml), methanol water 1:1 (50 ml) and methanol (50 ml) and dried to afford the title compound (0.68 g) as a brown solid. ¹H NMR (300 MHz, DMSO_{d6}) δ 6.88 (d, 2H), 7.15-7.23 (m, 2H), 7.50 (d, 1H), 7.64 (d, 2H), 7.70-7.76 (m, 3H), 9.42 (s, 1H), 9.60 (s, 1H). ¹⁹F NMR (282 MHz, DMSO_{d6}) δ− 112.3. MS (API-ES, M+H) 392.9. M. pt. 276° C. dec.

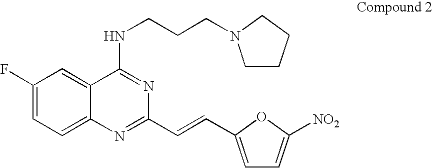

(E)-6-fluoro-2-(2-(5-nitrofuran-2-yl)vinyl)-N-(3-(pyrrolidin-1-yl)propyl)quinazolin-4-amine: This compound was prepared in the same manner as compound 1 but using 1-(3-aminopropyl)pyrrolidine instead of 4-aminophenol in the final step. ¹H NMR (300 MHz, TFA) 62.15 (m, 4H), 3.38 (m, 2H), 3.12 (m, 2H), 3.45 (m, 2H), 3.82 (m, 2H), 4.08 (m, 2H), 7.02 (d, 1H), 7.30 (d, 1H), 7.48 (m, 1H), 7.72-7.83 (m, 3H), 8.06 (d, 1H). ¹⁹F NMR (282 MHz, DMSO_{d6}) δ −107.1. MS (API-ES, M+H) 412.2. M. pt. 239-242° C. dec.

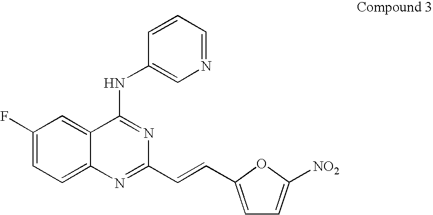

(E)-6-fluoro-2-(2-(5-nitrofuran-2-yl)vinyl)-N-(pyridin-3-yl)quinazolin-4-amine: This compound was prepared in the same manner as compound 1 but using 3-aminopyridine instead of 4-aminophenol in the final step. ¹H NMR (300 MHz, DMSO_{d6}) δ 7.24 (d, 1H), 7.33 (d, 1H), 7.65 (d, 1H), 7.78 (d, 1H), 7.82-7.90 (m, 3H), 8.58 (m, 2H), 8.73 (d, 1H), 9.51 (d, 1H), 10.63 (bs, 1H). ¹⁹F NMR (282 MHz, DMSO_{d6}) δ −111.5. MS (API-ES, M+H) 378.2. M. pt. 300° C. dec.

### EXAMPLE IX

### Compound 4

Bis-(2-trimethylsilanyloxy-ethyl)-amine: To a solution of diethanolamine (21 g) and thriethylamine (70 ml) in anhydrous THF (300 ml) at 0° C., was added dropwise chlorotrimethylsilane (55.6 ml). The mixture was stirred overnight at room temperature and then quenched with water (500 ml). The reaction mixture was extracted with ethyl acetate (3×300 ml). The combined organic layers were dried (Na₂SO₄) and concentrated in vacuo to afford the title compound (42 g) as a clear oil.

{6-Fluoro-2-[2-(5-nitro-furan-2-yl)-vinyl]-quinazolin-4-yl}-bis-(2-trimethylsilanyloxy-ethyl)-amine: To a suspension of 4-Chloro-6-fluoro-2-[2-(5-nitrofuran-2-yl)-vinyl]quinazoline (1 g) in 1-methyl-2-pyrrolidinone (10 ml), was added bis-(2-trimethylsilanyloxy-ethyl)-amine (2 g). The mixture was heated to 90° C. for 1 h and quenched by pouring into water (200 ml). The precipitate was filtered, washed with water and dried at 60° C. to give a crude solid. Further purification by silica gel chromatography (eluent ethyl acetate/hexane 1:1) gave the title compound (1 g) as a yellow solid.

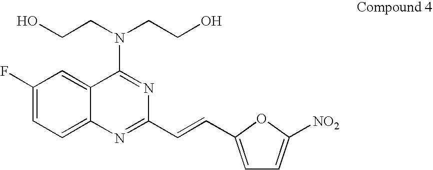

(E)-2,2′-(6-fluoro-2-(2-(5-nitrofuran-2-yl)vinyl)quinazolin-4-ylazanediyl)diethanol: 1N HCl (10 ml) was added to a solution of {6-Fluoro-2-[2-(5-nitro-furan-2-yl)-vinyl]-quinazolin-4-yl}-bis-(2-trimethylsilanyloxy-ethyl)-amine (0.9 g) in methanol (20 ml). The reaction mixture was stirred at ambient temperature for 2 h. The solvent was removed in vacuo to afford the title compound as a brown solid. ¹H NMR (300 MHz, DMSO_{d6}) δ 3.91 (m, 2H), 4.11 (m, 2H), 7.30 (d, 1H), 7.38 (d, 1H), 7.83 (d, 1H), 7.96 (m, 2H), 8.06 (d, 1H), 8.58 (d, 1H). ¹⁹F NMR (282 MHz, DMSO_{d6}) δ −111.2. MS (API-ES, M+H) 389.1. M. pt. 300° C. dec.

### EXAMPLE X

### Compounds 5 to 7

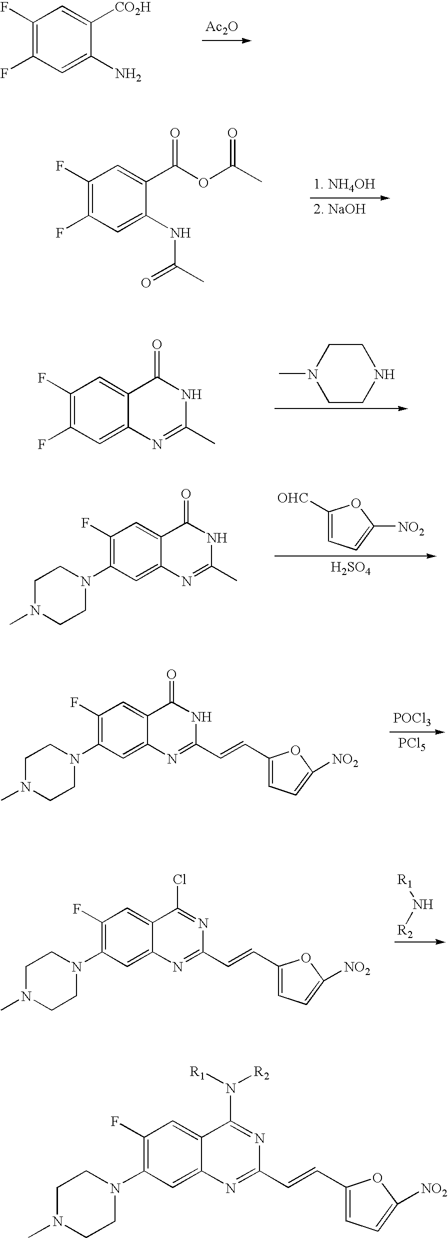

6,7-Difluoro-2-methyl-3H-quinazolin-4-one: A suspension of 2-amino-4,5-difluorobenzoic acid (55 g) in acetic anhydride (500 ml) was heated to reflux for 4 h. The acetic anhydride was removed in vacuo to give an off-white solid. Concentrated ammonium hydroxide (550 ml) was slowly added to the solid and the resulting mixture was stirred overnight at ambient temperature. Sodium hydroxide 10% w/v (220 ml) was added to the reaction mixture and the whole was heated to reflux for 1 h. The mixture was cooled to ambient temperature and adjusted to pH 8 with conc. HCl. The precipitate was filtered, washed with a small amount of water and dried at 60° C. overnight to give the title compound (54 g) as an off-white solid.

6-Fluoro-2-methyl-7-(4-methylpiperazin-1-yl)-3H-quinazolin-4-one: 1-methylpiperazine (40.1 g) was added to a suspension of 6,7-Difluoro-2-methyl-3H-quinazolin-4-one (19.6 g) in dimethylsulfoxide (170 ml) and the mixture was heated to 80° C. overnight. The precipitate was collected by filtration, washed with water and dried at 70° C. overnight to give the title compound (22 g) as an off-white solid.

6-Fluoro-7-(4-methylpiperazin-1-yl)-2-[2-(5-nitrofuran-2-yl)vinyl]-3H-quinazolin-4-one: 5-nitrofuraldehyde (6.3 g) and sulfuric acid (0.6 ml) were added to a solution of 6-Fluoro-2-methyl-7-(4-methylpiperazin-1-yl)-3H-quinazolin-4-one (8.28 g) in acetic acid (60 ml) and the mixture was heated to reflux for 24 h. The mixture was cooled to ambient temperature and ethyl acetate (200 ml) was added. The precipitate was filtered, washed with ethyl acetate and dried at 60° C. to give the title compound quantitative yield as a brown solid.

4-Chloro-6-fluoro-7-(4-methylpiperazin-1-yl)-2-[2-(5-nitrofuran-2-yl)vinyl]quinazoline: Phosphorus pentachloride (11.1 g) was added to a suspension of 6-Fluoro-7-(4-methylpiperazin-1-yl)-2-[2-(5-nitrofuran-2-yl)vinyl]-3H-quinazolin-4-one (all of the above) in phosphorus oxychloride (122.2 ml) and the mixture was heated to reflux for 24 h. The mixture was colled to ambient temperature and ethyl ether was added (400 ml). The precipitate was filtered, washed with ether and dried to give the title compound in quantitative yield as a tan solid.

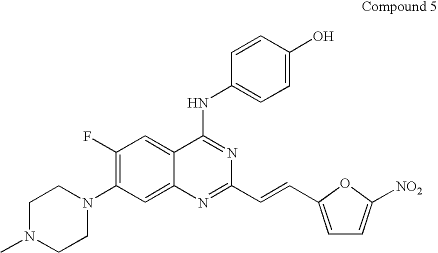

(E)-4-(6-fluoro-7-(4-methylpiperazin-1-yl)-2-[2-(5-nitrofuran-2-yl)vinyl]quinazolin-4-ylamino)phenol: This compound was prepared in the same manner as (E)-4-(6-fluoro-2-(2-(5-nitrofuran-2-yl)vinyl)quinazolin-4-ylamino)phenol (compound 1) but starting with 4-Chloro-6-fluoro-7-(4-methylpiperazin-1-yl)-2-[2-(5-nitrofuran-2-yl)vinyl]quinazoline to give a brown solid. ¹H NMR (300 MHz, TFA) δ 3.19 (s, 3H), 3.54 (m, 2H), 3.65 (m, 2H), 3.92 (m, 2H), 4.16 (m, 2H), 7.02 (s, 1H), 7.19 (d, 2H), 7.41 (m, 2H), 7.59 (m, 3H), 7.95 (d, 1H), 8.13 (d, 1H). MS (API-ES, M+H) 491.2. M. pt. 300° C. dec.

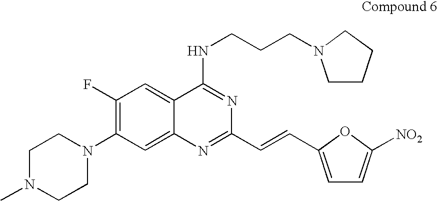

(E)-6-fluoro-7-(4-methylpiperazin-1-yl)-2-(2-(5-nitrofuran-2-yl)vinyl)-N-(3-(pyrrolidin-1-yl)propyl)quinazolin-4-amine: This compound was prepared in the same manner as compound 2 but using 4-Chloro-6-fluoro-7-(4-methylpiperazin-1-yl)-2-[2-(5-nitrofuran-2-yl)vinyl]quinazoline instead of 4-Chloro-6-fluoro-2-[2-(5-nitrofuran-2-yl)-vinyl]quinazoline. ¹H NMR (300 MHz, CD₂Cl₂) δ 1.97 (m, 6H). 2.33 (s, 3H), 2.58 (m, 4H), 2.68 (m, 4H), 2.84 (m, 2H), 3.25 (m, 4H), 3.78 (m, 2H), 6.74 (d, 1H), 7.19 (d, 1H), 7.21 (d, 1H), 7.30 (d, 1H), 7.39 (d, 1H), 7.76 (d, 1H), 8.88 (bs, 1H). ¹⁹F NMR (282 MHz, CD₂Cl₂) δ −122.2. MS (API-ES, M+H) 510.3. M. pt. 300° C. dec.

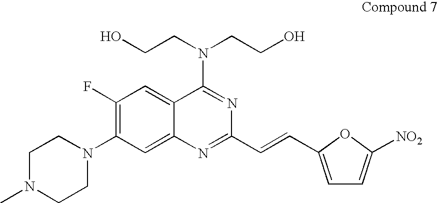

(E)-2,2′-(6-fluoro-7-(4-methylpiperazin-1-yl)-2-(2-(5-nitrofuran-2-yl)vinyl)quinazolin-4-ylazanediyl)diethanol: This compound was prepared in the same manner as in Example IX for compound 4 but using 4-Chloro-6-fluoro-7-(4-methylpiperazin-1-yl)-2-[2-(5-nitrofuran-2-yl)vinyl]quinazoline instead of 4-Chloro-6-fluoro-2-[2-(5-nitrofuran-2-yl)-vinyl]quinazoline. ¹H NMR (300 MHz, DMSO_{d6}) δ 2.22 (s, 3H), 2.75 (m, 2H), 3.06 (m, 2H), 3.19 (m, 4H), 3.42 (m, 2H), 3.53 (m, 4H), 4.62 (m, 2H), 7.09-7.20 (m, 3H), 7.64 (d, 1H), 7.70 (m, 2H). ¹⁹F NMR (282 MHz, DMSO_{d6}) δ −118.2. MS (API-ES, M+H) 487.1. M. pt. 227° C. dec.

### EXAMPLE XI

### Compounds 8 to 13

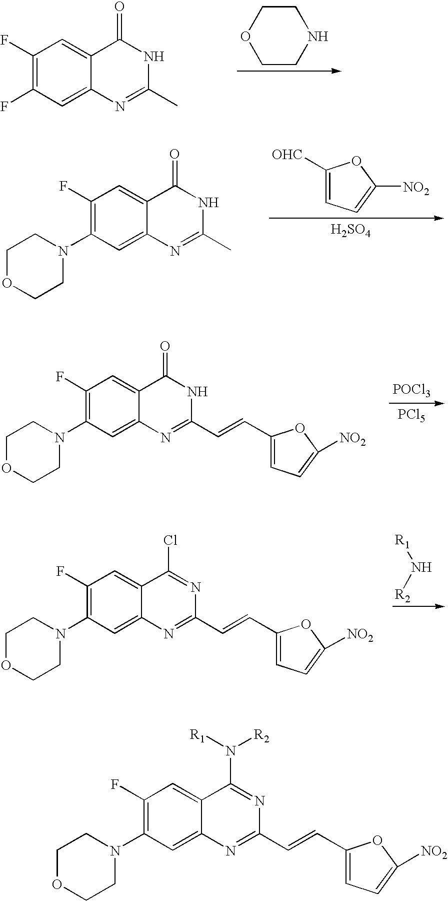

6-Fluoro-2-methyl-7-morpholin-4-yl-3H-quinazolin-4-one: This compound was prepared in the same manner as 6-Fluoro-2-methyl-7-(4-methylpiperazin-1-yl)-3H-quinazolin-4-one from 6,7-Difluoro-2-methyl-3H-quinazolin-4-one but using morpholine instead of 1-methylpiperazine.

6-fluoro-7-morpholin-4-yl-2-[2-(5-nitrofuran-2-yl)vinyl]-3H-quinazolin-4-one and 4-chloro-6-fluoro-7-morpholin-4-yl-2-[2-(5-nitrofuran-2-yl)vinyl]quinazoline were prepared in the same manner as 6-fluoro-7-(4-methylpiperazin-1-yl)-2-[2-(5-nitrofuran-2-yl)vinyl]-3H-quinazolin-4-one and 4-chloro-6-fluoro-7-(4-methylpiperazin-1-yl)-2-[2-(5-nitrofuran-2-yl)vinyl]quinazoline, respectively.

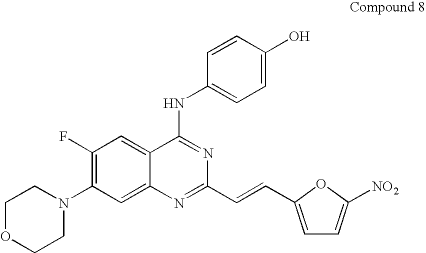

(E)-4-(6-fluoro-7-morpholino-2-(2-(5-nitrofuran-2-yl)vinyl)quinazolin-4-ylamino)phenol: This compound was prepared in the same manner as (E)-4-(6-fluoro-2-(2-(5-nitrofuran-2-yl)vinyl)quinazolin-4-ylamino)phenol (compound 1) but starting with 4-chloro-6-fluoro-7-morpholin-4-yl-2-[2-(5-nitrofuran-2-yl)vinyl]quinazoline to give a brown solid. ¹H NMR (300 MHz, TFA) 63.87 (m, 4H), 4.30 (m, 4H), 6.98 (m, 1H), 7.13 (d, 2H), 7.30 (d, 1H), 7.48-7.57 (m, 3H), 7.81 (m, 1H), 7.91 (d, 1H), 8.26 (d, 1H). MS (API-ES, M+H) 478.1. M. pt. 300° C. dec.

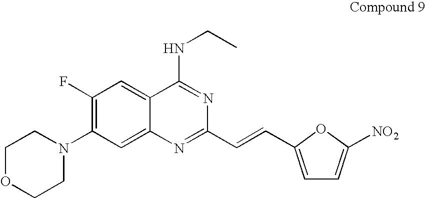

(E)-N-ethyl-6-fluoro-7-morpholino-2-(2-(5-nitrofuran-2-yl)vinyl)quinazolin-4-amine: This compound was prepared in the same manner as compound 1 but starting with 4-chloro-6-fluoro-7-morpholin-4-yl-2-[2-(5-nitrofuran-2-yl)vinyl]quinazoline and using ethyl amine instead of 4-aminophenol. ¹H NMR (300 MHz, DMSO_{d6}) δ 1.27 (t, 3H), 3.18 (m, 4H), 3.63 (m, 2H), 3.79 (m, 4H), 7.14 (d, 1H), 7.19 (d, 1H), 7.26 (d, 1H), 7.73 (d, 1H), 7.81 (d, 1H), 8.06 (d, 1H). ¹⁹F NMR (282 MHz, DMSO_{d6}) δ −121.6. MS (API-ES, M+H) 414.3.

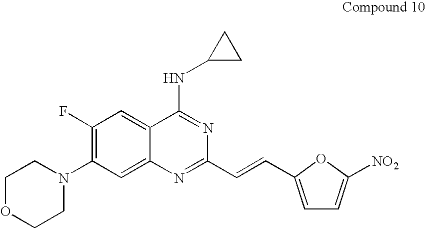

(E)-N-cyclopropyl-6-fluoro-7-morpholino-2-(2-(5-nitrofuran-2-yl)vinyl)quinazolin-4-amine: This compound was prepared in the same manner as compound 1 but starting with 4-chloro-6-fluoro-7-morpholin-4-yl-2-[2-(5-nitrofuran-2-yl)vinyl]quinazoline and using cyclopropyl amine instead of 4-aminophenol. ¹H NMR (300 MHz, DMSO_{d6}) δ 0.67 (m, 2H), 0.86 (m, 2H), 3.16 (m, 4H), 3.32 (m, 1H), 3.78 (m, 4H), 7.16-7.26 (m, 2H), 7.77 (d, 1H), 7.80 (s, 1H), 8.01 (s, 1H), 8.03 (d, 1H). ¹⁹F NMR (282 MHz, DMSO_{d6}) δ −121.7. MS (API-ES, M+H) 426.0.

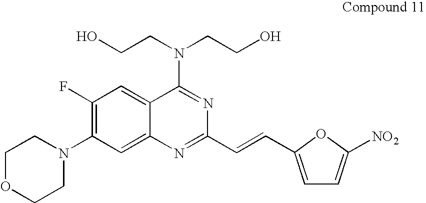

(E)-2,2′-(6-fluoro-7-morpholino-2-(2-(5-nitrofuran-2-yl)vinyl)quinazolin-4-ylazanediyl)diethanol This compound was prepared in the same manner as compound 4 but starting with from 4-chloro-6-fluoro-7-morpholin-4-yl-2-[2-(5-nitrofuran-2-yl)vinyl]quinazoline and using Bis-(2-trimothylsilanyloxy-ethyl)-amine instead of 4-aminophenol. ¹H NMR (300 MHz, DMSO_{d6}) δ 3.19 (m, 4H), 3.80 (m, 10H), 5.04 (m, 2H), 7.15-7.26 (m, 3H), 7.79 (d, 1H), 7.82 (d, 1H), 8.20 (d, 1H). ¹⁹F NMR (282 MHz, DMSO_{d6}) δ −121.5. MS (API-ES, M+H) 474.3.

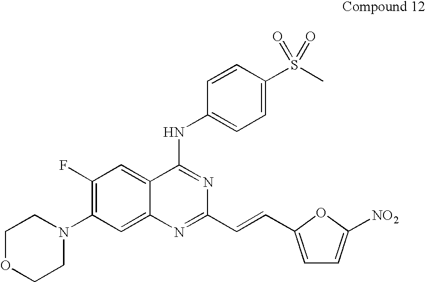

(E)-6-fluoro-N-(4-(methylsulfonyl)phenyl)-7-morpholino-2-(2-(5-nitrofuran-2-yl)vinyl)quinazolin-4-amine: This compound was prepared in the same manner as compound 1 but starting with 4-chloro-6-fluoro-7-morpholin-4-yl-2-[2-(5-nitrofuran-2-yl)vinyl]quinazoline and using 4-(methylsulfonyl)aniline instead of 4-aminophenol. ¹H NMR (300 MHz, DMSO_{d6}) δ 3.23 (m, 4H), 3.32 (s, 3H), 3.81 (m, 4H), 7.21-7.30 (m, 3H), 7.69 (d, 1H), 7.80 (m, 1H), 7.99 (d, 2H), 8.25 (d, 2H), 8.42 (d, 1H), 9.88 (s, 1H). ¹⁹F NMR (282 MHz, DMSO_{d6}) δ −119.5. MS (API-ES, M+H) 540.0.

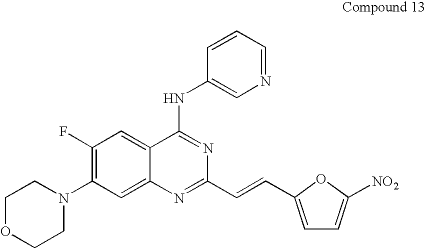

(E)-6-fluoro-7-morpholino-2-(2-(5-nitrofuran-2-yl)vinyl)-N-(pyridin-3-yl)quinazolin-4-amine: This compound was prepared in the same manner as compound 3 but starting with 4-chloro-6-fluoro-7-morpholin-4-yl-2-[2-(5-nitrofuran-2-yl)vinyl]quinazoline. ¹H NMR (300 MHz, DMSO_{d6}) δ 3.81 (m, 4H), 4.30 (m, 4H), 7.17 (d, 1H), 7.43 (d, 1H), 7.52 (d, 1H), 7.63 (d, 1H), 8.04 (d, 1H), 8.23 (d, 1H), 8.36 (m, 1H), 8.89 (m, 1H), 9.20 (d, 1H), 9.80 (s, 1H). MS (API-ES, M+H) 463.1.

### EXAMPLE XII

### Compound 14

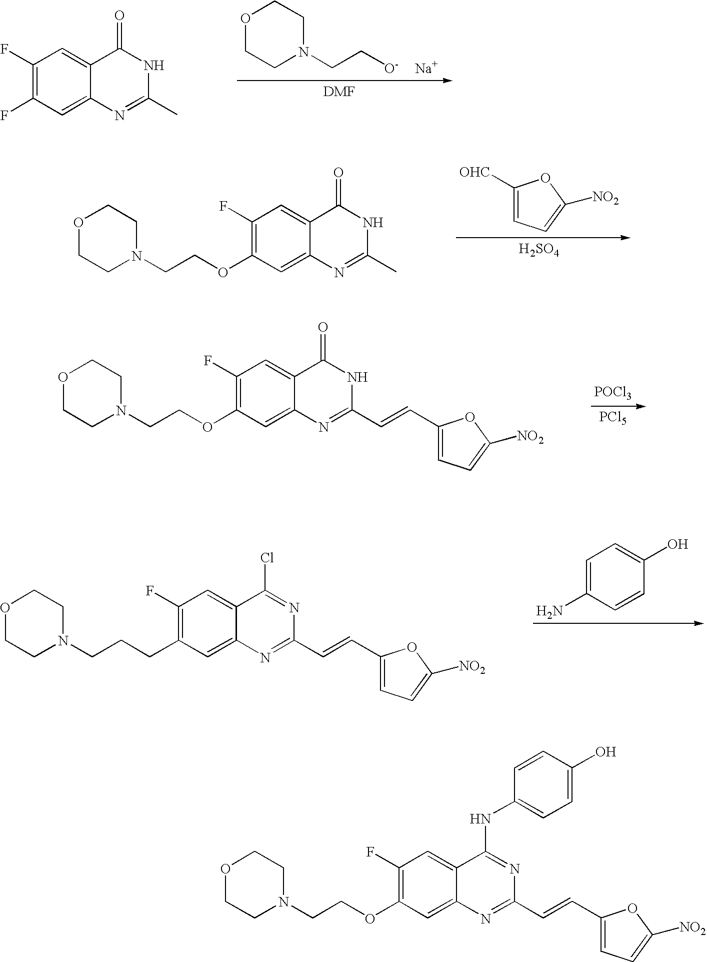

6-Fluoro-2-methyl-7-(2-morpholin-4-yl-ethoxy)-3H-quinazolin-4-one: Sodium hydride (8.0 g of a 60% dispersion in mineral oil) was added to a solution of 4-(2-hydroxyethyl)-morpholine (24.5 ml) in dimethylformamide (100 ml) at 0° C. The resulting mixture was stirred at that temperature for 30 min. 6-Fluoro-2-methyl-7-morpholin-4-yl-3H-quinazolin-4-one (9.8 g) was added and the reaction mixture was heated to 90° C. for 3 h. The reaction was cooled to 0° C. and quenched with water. The mixture was concentrated in vacuo and the residue taken up in water and extracted with ethyl acetate. The combined organic layers were dried (Na₂SO₄) and concentrated to afford the title compound (13.64 g) as an off-white solid.

6-Fluoro-7-(2-morpholin-4-yl-ethoxy)-2-[2-(5-nitrofuran-2-yl)vinyl]-3H-quinazolin-4-one: To a solution of 6-Fluoro-2-methyl-7-(2-morpholin-4-yl-ethoxy)-3H-quinazolin-4-one (13.64 g) in acetic acid (200 ml) was added 5-nitro-2-furaldehyde (9.4 g) and conc. sulfuric acid (0.5 ml) and the reaction mixture was heated to reflux for 2 h. The reaction mixture was cooled to ambient temperature and ethyl acetate (200 ml) was added. The precipitate was filtered, washed with ethyl acetate and dried at 60° C. to give the title compound as a dark solid.

4-Chloro-6-Fluoro-7-(2-morpholin-4-yl-ethoxy)-2-[2-(5-nitrofuran-2-yl)vinyl]-quinazoline: To a suspension of 6-Fluoro-7-(2-morpholin-4-yl-ethoxy)-2-[2-(5-nitrofuran-2-yl)vinyl]-3H-quinazolin-4-one (6.1 g) in phosphorus oxychloride (100 ml), was added phosphorus pentachloride (6 g) and the reaction mixture was heated to reflux for 24 h. The reaction mixture was cooled to ambient temperature and ethyl ether (200 ml) was added. The precipitate was filtered, washed with ether and dried to give the title compound (6.2 g) as a dark brown solid.

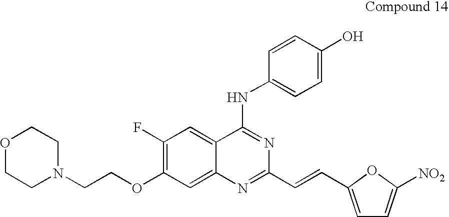

(E)-4-(6-fluoro-7-(2-morpholinoethoxy)-2-(2-(5-nitrofuran-2-yl)vinyl)quinazolin-4-ylamino)phenol: To a solution of 4-Chloro-6-Fluoro-7-(2-morpholin-4-yl-ethoxy)-2-[2-(5-nitrofuran-2-yl)vinyl]-quinazoline (6.2 g) in dimethylformamide (50 ml) was added 4-aminophenol (3.77 g) and the resulting mixture was heated to 90° C. for 9 h. The mixture was cooled to ambient temperature and poured into water (200 ml). The precipitate was filtered, washed with water, then methanol and dried at 60° C. under vacuum to give a crude solid that was further purified by silica gel column chromatography (ethyl acetate/methanol/triethylamine 85:10:5) to give the title compound (0.95 g) as a tan solid. ¹H NMR (300 MHz, DMSO_{d6}) δ 2.55 (m, 4H), 2.78 (m, 2H), 3.59 (m, 4H), 4.32 (bt, 2H), 6.82 (d, 2H), 7.15 (d, 1H), 7.23 (d, 1H), 7.33 (d, 1H), 7.53 (d, 1H), 7.58 (d, 2H), 7.76 (d, 1H), 8.34 (d, 1H), 9.33 (s, 1H), 9.42 (s, 1H). ¹⁹F NMR (282 MHz, DMSO_{d6}) δ −131.9. MS (API-ES, M+H) 522.2. M. pt. 235° C. dec.

### EXAMPLE XIII

### Compounds 15 to 21

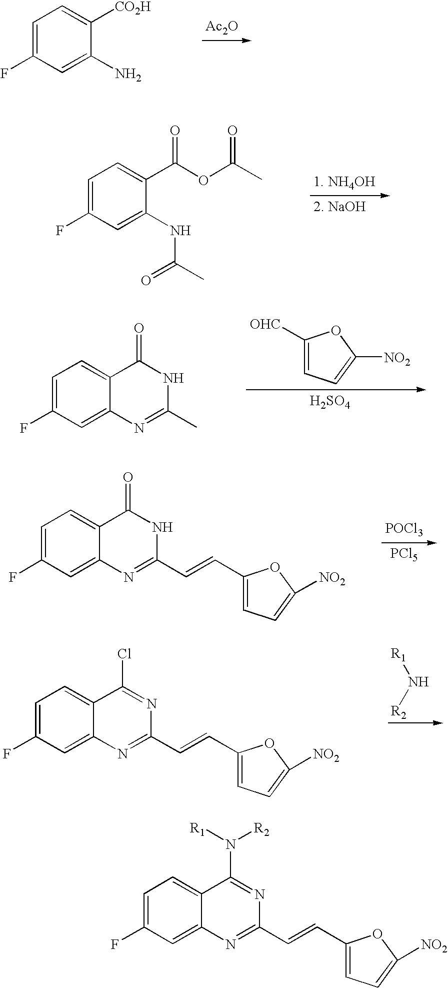

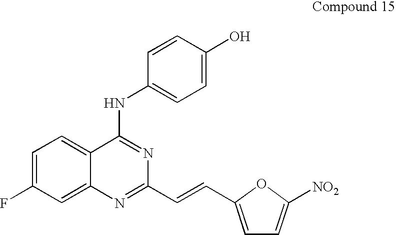

(E)-4-(7-fluoro-2-(2-(5-nitrofuran-2-yl)vinyl)quinazolin-4-ylamino)phenol: This compound was prepared in the same manner as compound 1 but using 2-amino-4-fluorobenzoic acid instead of 2-amino-5-fluorobenzoic acid. ¹H NMR (300 MHz, DMSO_{d6}) δ 6.85 (d, 1H), 7.21 (d, 1H), 7.32 (d, 1H), 7.50 (d, 1H), 7.60 (m, 5H), 7.80 (d, 1H), 8.51 (m, 1H), 9.40 (s, 1H), 9.79 (s, 1H). ¹⁹F NMR (282 MHz, DMSO_{d6}) δ −106.4. MS (API-ES, M+H) 392.9. M. pt. 265° C. dec.

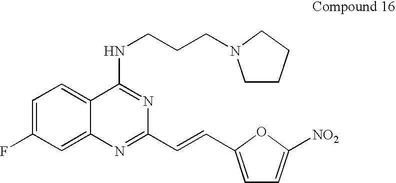

(E)-7-fluoro-2-(2-(5-nitrofuran-2-yl)vinyl)-N-(3-(pyrrolidin-1-yl)propyl)quinazolin-4-amine: This compound was prepared in the same manner as compound 15 but using 1-(3-aminopropyl)pyrrolidine instead of 4-aminophenol in the final step. ¹H NMR (300 MHz, TFA) 61.99 (m, 4H), 2.25 (m, 2H), 2.96 (m, 2H), 3.30 (m, 2H), 3.68 (m, 2H), 3.92 (m, 2H), 6.86 (s, 1H), 7.13 (d, 1H), 7.30 (m, 3H), 7.90 (d, 1H), 8.03 (m, 1H). ¹⁹F NMR (282 MHz, TFA) δ −96.3. MS (API-ES, M+H) 412.4. M. pt. 300° C. dec.

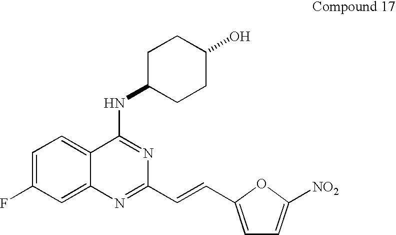

4-(7-fluoro-2-((E)-2-(5-nitrofuran-2-yl)vinyl)quinazolin-4-ylamino)cyclohexanol: This compound was prepared in the same manner as compound 15 but using trans 4-aminocyclohexanol instead of 4-aminophenol in the final step. ¹H NMR (300 MHz, DMSO_{d6}) δ 1.42 (m, 4H), 1.96 (m, 4H), 3.48 (m, 1H), 4.23 (m, 1H), 4.60 (s, 1H), 7.20 (d, 1H), 7.29 (d, 1H), 7.40 (m, 2H), 7.74 (d, 2H), 7.80 (d, 1H), 8.02 (d, 1H), 8.41 (m, 1H). ¹⁹F NMR (282 MHz, DMSO_{d6}) δ −107.5. MS (API-ES, M+H) 399.0.

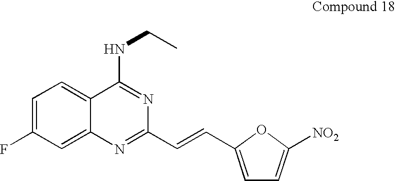

(E)-N-ethyl-7-fluoro-2-(2-(5-nitrofuran-2-yl)vinyl)quinazolin-4-amine: This compound was prepared in the same manner as compound 15 but using ethylamine instead of 4-aminophenol in the final step. ¹H NMR (300 MHz, DMSO_{d6}) 61.29 (t, 3H), 3.63 (m, 2H), 7.20 (d, 1H), 7.26 (d, 1H), 7.40 (m, 2H), 7.75 (d, 1H), 7.79 (s, 1H), 8.30 (m, 1H), 8.37 (m, 1H). ¹⁹F NMR (282 MHz, DMSO_{d6}) δ −107.5. MS (API-ES, M+H) 329.1.

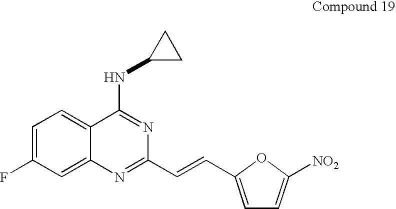

(E)-N-cyclopropyl-7-fluoro-2-(2-(5-nitrofuran-2-yl)vinyl)quinazolin-4-amine: This compound was prepared in the same manner as compound 15 but using cyclopropylamine instead of 4-aminophenol in the final step. ¹H NMR (300 MHz, DMSO_{d6}) δ 0.71 (m, 2H), 0.89 (m, 2H), 3.21 (m, 1H), 7.26 (m, 2H), 7.43 (m, 2H), 7.79 (s, 1H), 7.81 (d, 1H), 8.31 (m, 1H), 8.38 (m, 1H). ¹⁹F NMR (282 MHz, DMSO_{d6}) δ− 106.9. MS (API-ES, M+H) 341.1.

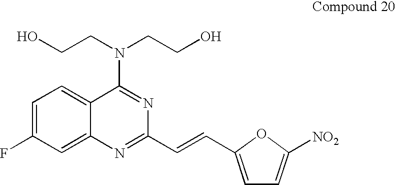

(E)-2,2′-(7-fluoro-2-(2-(5-nitrofuran-2-yl)vinyl)quinazolin-4-ylazanediyl)diethanol: This compound was prepared in the same manner as compound 4 but using 4-Chloro-7-fluoro-2-[2-(5-nitrofuran-2-yl)-vinyl]quinazoline instead of 4-Chloro-6-fluoro-2-[2-(5-nitrofuran-2-yl)vinyl]quinazoline. ¹H NMR (300 MHz, DMSO_{d6}) δ 3.91 (m, 4H), 4.09 (m, 4H), 7.28 (d, 1H), 7.38 (d, 1H), 7.56 (m, 1H), 7.68 (d, 1H), 7.81 (d, 1H), 8.07 (d, 1H), 8.71 (m, 1H). ¹⁹F NMR (282 MHz, DMSO_{d6}) δ −101.9. MS (API-ES, M+H) 389.1.

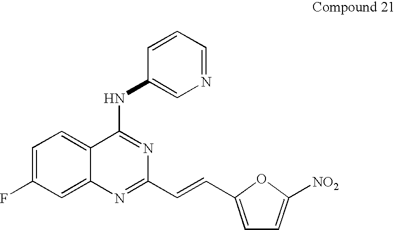

(E)-7-fluoro-2-(2-(5-nitrofuran-2-yl)vinyl)-N-(pyridin-3-yl)quinazolin-4-amine: This compound was prepared in the same manner as example 15 but using 3-aminopyridine instead of 4-aminophenol in the final step. ¹H NMR (300 MHz, DMSO_{d6}) δ 7.29 (d, 1H), 7.31 (s, 1H), 7.50-7.64 (m, 4H), 7.80 (d, 1H), 8.37 (d, 1H), 8.40 (d, 1H), 8.64 (m, 1H), 9.05 (d, 1H), 10.1 (s, 1H). ¹⁹F NMR (282 MHz, DMSO_{d6}) δ −105.5. MS (API-ES, M+H) 378.0. M. pt. 277.7-278.4° C. dec.

### EXAMPLE XIV

### Compounds 22 to 28

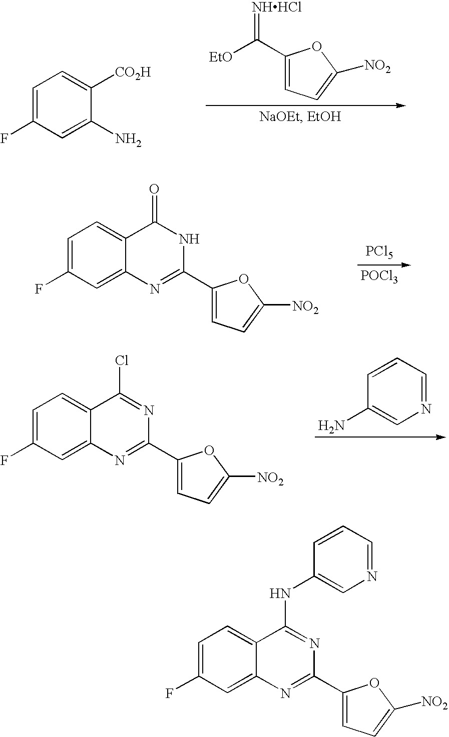

7-Fluoro-2-(5-nitro-furan-2-yl)-3H-quinazolin-4-one. To a stirred solution of sodium ethoxide (37 ml) in ethanol (300 ml) was added ethyl 5-nitro-2-furimidate hydrochloride (22 g) and then 2-amino-4-fluorobenzoic acid (15.5 g). The mixture was refluxed overnight, cooled to room temperature and concentrated to dryness. Ice water (1 l) was added to the residue and the resulting mixture was acidified with acetic acid (pH 3-4). The crude product was filtered, washed with water (500 ml) and ethyl acetate (1 l) to afford the title compound as an off-white solid.

4-Chloro-7-fluoro-2-(5-nitro-furan-2-yl)quinazoline. To a stirred solution of PCl₅ (2.4 g) in POCl₃ (26 ml) was added 7-fluoro-2-(5-nitro-furan-2-yl)-3H-quinazolin-4-one (2.76 g). The mixture was refluxed for 2 hours, cooled to room temperature and poured into ether (200 ml). The product was collected by filtration and washed with ether. The crude product (light brown solid) was used for next step without further purification.

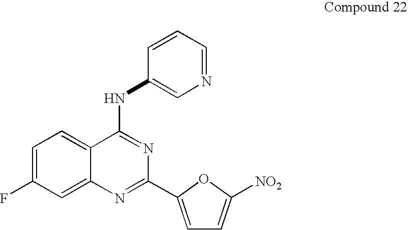

[7-Fluoro-2-(5-nitrofuran-2-yl)-quinazolin-4-yl]-pyridin-3-yl-amine. To a solution of 4-Chloro-7-fluoro-2-(5-nitro-furan-2-yl)-quinazoline (0.88 g) in DMF (15 ml) was added 3-aminopyridine (0.73 g). The mixture was heated to 90° C. for 2 hours, cooled to room temperature and poured into icy water (200 ml). The mixture was then extracted with THF and concentrated to dryness. The residue was purified by column chromatography (THF/Heptane, 1:1) on silica gel to afford the title compound as yellow solid. ¹H NMR (300 MHz, DMSO_{d6}) δ 7.42 (d, 1H), 7.49 (dd, 1H), 7.51-7.75 (m, 2H), 7.82 (d, 1H), 8.34-8.45 (m, 2H), 8.70 (dd, 1H), 8.07 (d, 1H), 10.23 (s, 1H). ¹⁹F NMR (282 MHz, DMSO_{d6}) δ −104.7. MS (API-ES, M+H) 352.21. M. pt. 292-293° C. dec.

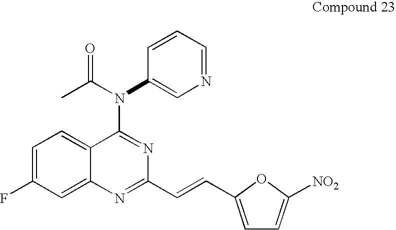

(E)-N-(7-fluoro-2-(2-(5-nitrofuran-2-yl)vinyl)quinazolin-4-yl)-N-(pyridin-3-yl)acetamide: (E)-7-fluoro-2-(2-(5-nitrofuran-2-yl)vinyl)-N-(pyridin-3-yl)quinazolin-4-amine in pyridine was treated with acetic anhydride in the presence of catalytic amount of DMAP at 60° C. to afford the title compound in 80% yield. Chromatography by DCM/THF gave the title compound (0.48 g). ¹H NMR (300 MHz, DMSO_{d6}) 6.2.18 (s, 3H), 7.37 (d, 1H), 7.43 (d, 1H), 7.51 (m, 1H), 7.66 (m, 1H), 7.81-7.87 (m, 2H), 7.90 (d, 1H), 8.02 (bd, 1H), 8.33 (m, 1H), 8.55 (m, 1H), 8.83 (d, 1H). ¹⁹F NMR (282 MHz, DMSO_{d6}) δ −102.5. MS (API-ES, M+H) 420.0. M. pt. 256-257° C. dec.

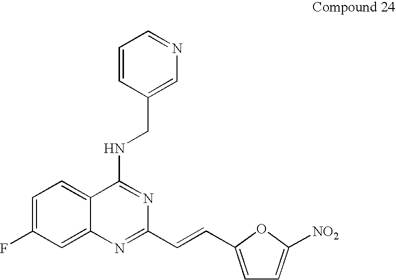

(E)-7-fluoro-2-(2-(5-nitrofuran-2-yl)vinyl)-N-(pyridin-3-ylmethyl)quinazolin-4-amine: This compound was prepared in the same manner as compound 15 but using pyridin-3-ylmethylamine instead of 4-aminophenol in the final step. ¹H NMR (300 MHz, TFA) 65.52 (s, 2H), 7.08 (d, 1H), 7.38 (d, 1H), 7.57 (m, 3H), 8.08 (d, 1H), 8.20 (m, 1H), 8.34 (m, 1H), 8.86 (m, 2H), 9.11 (s, 1H). MS (API-ES, M+H) 392.1. M. pt. 300° C. dec.

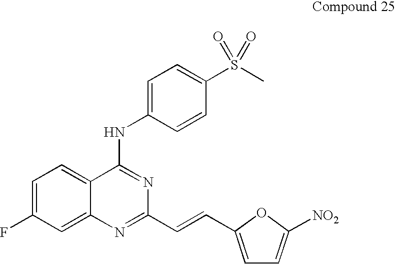

(E)-7-fluoro-N-(4-(methylsulfonyl)phenyl)-2-(2-(5-nitrofuran-2-yl)vinyl)quinazolin-4-amine: This compound was prepared in the same manner as compound 15 but using 4-(methylsulfonyl)aniline instead of 4-aminophenol in the final step. ¹H NMR (300 MHz, DMSO_{d6}) δ 3.25 (s, 3H), 7.26 (d, 1H), 7.38 (d, 1H), 7.61 (m, 2H), 7.75 (d, 1H), 7.81 (d, 1H), 8.03 (d, 2H), 8.26 (d, 2H), 8.80 (m, 1H), 10.6 (s, 1H). ¹⁹F NMR (282 MHz, DMSO_{d6}) δ −103.8. MS (API-ES, M+H) 455.1.

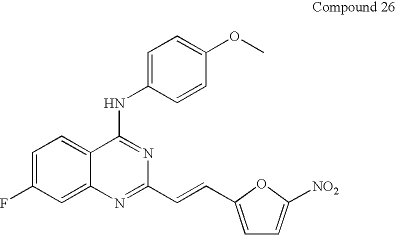

(E)-7-fluoro-N-(4-methoxyphenyl)-2-(2-(5-nitrofuran-2-yl)vinyl)quinazolin-4-amine: This compound was prepared in the same manner as compound 15 but using 4-methoxyaniline instead of 4-aminophenol in the final step. ¹H NMR (300 MHz, DMSO_{d6}) δ 3.81 (s, 3H), 7.02 (d, 2H), 7.20 (d, 1H), 7.29 (m, 1H), 7.52 (m, 2H), 7.60 (d, 1H), 7.78 (m, 3H), 8.61 (m, 1H), 9.87 (s, 1H). ¹⁹F NMR (282 MHz, DMSO_{d6}) δ −104.9. MS (API-ES, M+H) 407.1.

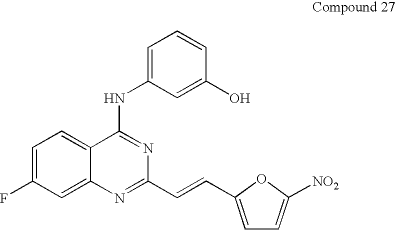

(E)-3-(7-fluoro-2-(2-(5-nitrofuran-2-yl)vinyl)quinazolin-4-ylamino)phenol: This compound was prepared in the same manner as compound 15 but using 3-aminophenol instead of 4-aminophenol in the final step. ¹H NMR (300 MHz, DMSO_{d6}) δ 6.59 (d, 1H), 7.31 (m, 4H), 7.56 (m, 3H), 7.69 (d, 1H), 7.80 (d, 1H), 8.66 (m, 1H), 9.81 (s, 1H). ¹⁹F NMR (282 MHz, DMSO_{d6}) δ −105.9. MS (API-ES, M+H) 393.0.

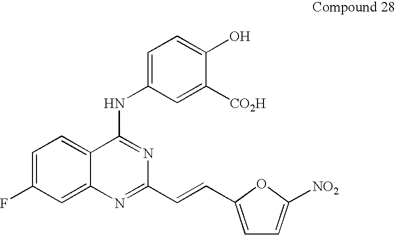

(E)-5-(7-fluoro-2-(2-(5-nitrofuran-2-yl)vinyl)quinazolin-4-ylamino)-2-hydroxybenzoic acid: This compound was prepared in the same manner as compound 15 but using 4-amino-2-hydroxybenzoic acid instead of 4-aminophenol in the final step. ¹H NMR (300 MHz, TFA) δ 7.18 (m, 1H), 7.42 (m, 2H), 7.70 (m, 3H), 8.02 (m, 1H), 8.22 (d, 1H), 8.62 (m, 1H), 8.86 (bs, 1H). MS (API-ES, M+H) 437.1.

### EXAMPLE XV

### Compound 29

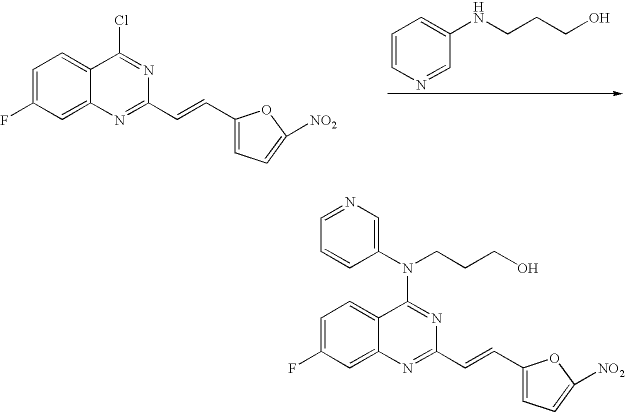

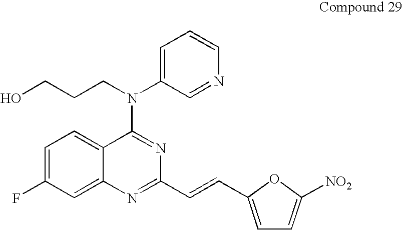

(E)-3-((7-fluoro-2-(2-(5-nitrofuran-2-yl)vinyl)quinazolin-4-yl)(pyridin-3-yl)amino)propan-1-ol: A suspension of 4-chloro-7-fluoro-2-(2-(5-nitrofuran-2-yl)vinyl)quinazoline (1.49 g) and 3-(pyridin-3-ylamino)propan-1-ol (1.49 g, 2.1 equiv.) in anhydrous acetonitrile (125 ml) was stirred under nitrogen at 80-82° C. for 5 hrs. The reaction mixture was allowed to cool overnight with stirring. The precipitate was collected by filtration and washed with acetonitrile. The crude material was suspended in dry THF (100 ml) and stirred at reflux under nitrogen for 20 min. After cooling to 35° C. the solid was collected by filtration, washed with THF, suspended in THF (100 m) and stirred overnight under nitrogen. The solid was collected by filtration, washed with THF and dried under vacuum at 35° C. to produce the title compound (1.44 g). ¹H NMR (300 MHz, DMSO_{d6}) δ 1.76 (m, 2H), 3.23 (m, 2H), 2.53 (t, 2H), 7.41 (d, 1H), 7.54 (d, 1H), 7.83 (m, 3H), 8.10 (m, 4H), 8.62 (m, 1H), 8.65 (s, 1H). ¹⁹F NMR (282 MHz, DMSO_{d6}) δ −98.8. MS (API-ES, M+H) 463.2. M. pt. 178.8-179.9° C. dec.

### EXAMPLE XVI

### Compounds 30 to 35

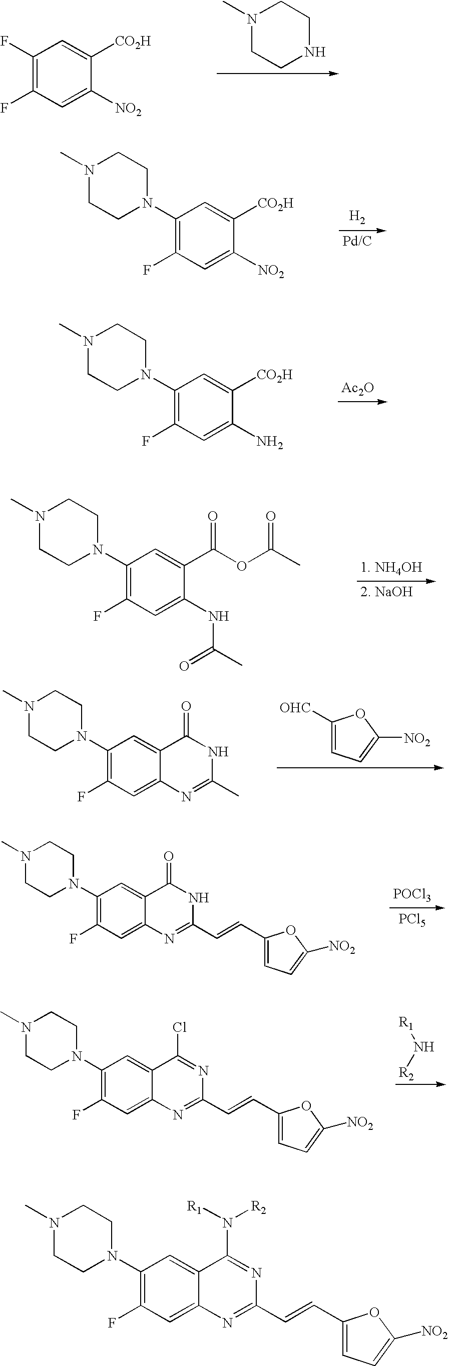

4-Fluoro-5-(4-methyl-piperazin-1-yl)-2-nitrobenzoic acid: A solution of 2-nitro-4,5-difluorobenzoic acid (20.2 g) and N-methyl-piperazine (55 ml) in dimethylsulfoxide (500 ml) was heated at 90° C. for 3 h. The solution was cooled to ambient temperature, diluted with water (1 l) and loaded onto amberlyst-15 ion exchange resin (1 kg). The desired product was eluted with ammonium hydroxide to afford a gummy solid (33 g). Trituration with ethyl acetate afforded the title compound (20.7 g) as a hygroscopic solid.

2-Amino-4-fluoro-5-(4-methyl-piperazin-1-yl)benzoic acid: A mixture of 4-fluoro-5-(4-methyl-piperazin-1-yl)-2-nitrobenzoic acid (20.7 g), palladium on carbon (2 g of a wet 50% suspension) and ethanol (500 ml) was shaken under 50 psi hydrogen atmosphere for 2 h. The mixture was filtered through Celite™ and concentrated in vacuo to afford the title compound (11 g) as a yellow foam.

7-Fluoro-2-methyl-6-(4-methyl-piperazin-1-yl)-3H-quinazolin-4-one: 2-Amino-4-fluoro-5-(4-methyl-piperazin-1-yl)-benzoic acid (11 g) in acetic anhydride (100 ml) was heated to reflux for 3 h. The mixture was concentrated in vacuo and treated with conc. Ammonium hydroxide (250 ml). The mixture was heated to reflux for 1 h and cooled to ambient temperature and brought to pH 8 by the addition of conc. HCl. The resulting precipitate was filtered and dried to afford the title compound (1 g) as a tan solid.

(E)-7-fluoro-6-(4-methylpiperazin-1-yl)-2-(2-(5-nitrofuran-2-yl)vinyl)quinazolin-4(3H)-one: A solution of 7-Fluoro-2-methyl-6-(4-methyl-piperazin-1-yl)-3H-quinazolin-4-one (1 g), 5-nitro-2-furaldehide (1 g) and conc. sulfuric acid (1 drop) in acetic acid (10 ml) was heated to 70° C. overnight. The mixture was cooled to ambient temperature and diluted with water (20 ml). The resulting precipitate was filtered, washed with water and dried to afford the title compound (1.3 g) as a brown solid.

(E)-4-chloro-7-fluoro-6-(4-methylpiperazin-1-yl)-2-(2-(5-nitrofuran-2-yl)vinyl)quinazoline: A solution of 7-fluoro-6-(4-methylpiperazin-1-yl)-2-(2-(5-nitrofuran-2-yl)vinyl)quinazolin-4(3H)-one (1.3 g) in phosphorus oxychloride (50 ml) was heated to reflux for 24 h. The mixture was concentrated in vacuo, co-evaporated with toluene. The resulting residue was treated with saturated aqueous sodium bicarbonate (200 ml) and extracted with ethyl acetate (3×100 ml). The insoluble solids were ground in a mortar and pestle to help dissolving. The combined organic layers were washed with brine, dried (Na₂SO₄) and dried further under reduced pressure to afford the title compound (320 mg) as a brown solid.

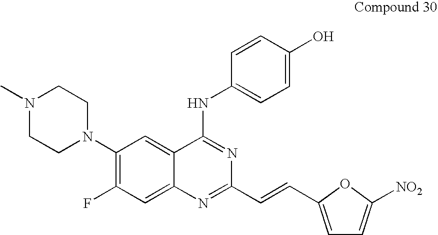

(E)-4-{7-fluoro-6-(4-methylpiperazin-1-yl)-2-(2-(5-nitrofuran-2-yl)vinyl)quinazolin-4-ylamino}phenol: A solution of (E)-4-chloro-7-fluoro-6-(4-methylpiperazin-1-yl)-2-(2-(5-nitrofuran-2-yl)vinyl)quinazoline (320 mg) and 4-aminophenol (209 mg) in 1-methyl-2-pyrrolidinone (15 ml) was heated at 90° C. for 2 h. The mixture was diluted with water (20 ml) and extracted with ethyl acetate/ethanol 9:1 (3×150 ml). The combined organic layers were washed with brine (100 ml), dried (Na₂SO₄) and concentrated in vacuo to afford a dark oil (2.5 g). Further purification by silica gel chromatography (dichloromethane with 0-10% methanol and 0-0.5% ammonium hydroxide gradients) and trituration with dichloromethane/methanol/ammonium hydroxide 95:5:0.2 afforded the title compound (180 mg) as an orange solid. ¹H NMR (300 MHz, DMSO_{d6}) δ 2.27 (s, 3H), 2.55 (m, 4H), 3.18 (m, 4H), 6.85 (d, 2H), 7.20 (d, 1H), 7.26 (d, 1H), 7.44-7.54 (m, 4H), 7.79 (d, 1H), 7.91 (d, 1H), 9.38 (2, 1H), 9.64 (s, 1H). ¹⁹F NMR (282 MHz, DMSO_{d6}) δ −114.4. MS (API-ES, M+H) 491.0.

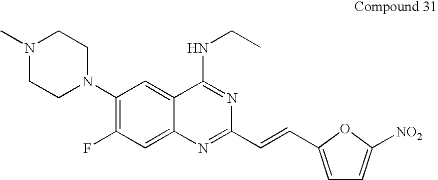

(E)-N-ethyl-7-fluoro-6-(4-methylpiperazin-1-yl)-2-(2-(5-nitrofuran-2-yl)vinyl)quinazolin-4-amine: This compound was prepared in the same manner as described for compound 30 but using ethanol amine instead of 4-aminophenol in the final step. ¹H NMR (300 MHz, TFA) δ 1.49 (t, 3H), 3.18 (s, 3H), 3.49 (m, 4H), 3.87 (m, 4H), 4.05 (m, 2H), 7.07 (d, 1H), 7.34 (d, 1H), 7.58 (m, 2H), 7.76 (d, 1H), 8.15 (d, 1H). MS (API-ES, M+H) 427.2. M. pt. 217° C. dec.

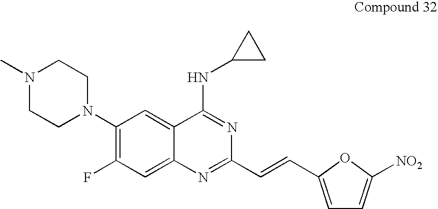

(E)-N-cyclopropyl-7-fluoro-6-(4-methylpiperazin-1-yl)-2-(2-(5-nitrofuran-2-yl)vinyl)quinazolin-4-amine: This compound was prepared in the same manner as described for compound 30 but using cyclopropyl amine instead of 4-aminophenol in the final step. ¹H NMR (300 MHz, DMSO_{d6}) δ 0.95 (m, 2H), 1.19 (m, 2H), 3.18 (m, 4H), 3.50 (m, 4H), 3.83 (m, 4H), 7.06 (s, 1H), 7.35 (d, 1H), 7.57 (m, 2H), 7.73 (d, 1H), 8.25 (d, 1H). MS (API-ES, M+H) 439.2. M. pt. 158.5-160.0° C. dec.

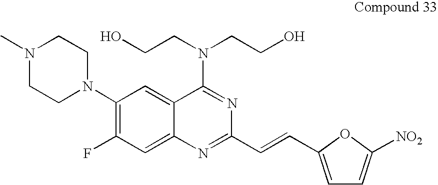

(E)-2,2′-(7-fluoro-6-(4-methylpiperazin-1-yl)-2-(2-(5-nitrofuran-2-yl)vinyl)quinazolin-4-ylazanediyl)diethanol: To a solution of (E)-4-chloro-7-fluoro-6-(4-methylpiperazin-1-yl)-2-(2-(5-nitrofuran-2-yl)vinyl)quinazoline (12.53 g) in 1-methyl-2-pyrrolidinone (80 ml) was added Bis-(2-trimothylsilanyloxy-ethyl)-amine (18.69 g) and the mixture was heated to 90° C. for 9 h. The reaction mixture was cooled to ambient temperature and poured into water (200 ml). The mixture was concentrated in vacuo and the residue taken up in methanol and treated with 1N HCl for 2 h. The mixture was concentrated in vacuo and the residual material washed with water followed by methanol, dried at 60° C. under vacuum to give a solid that was further purified by silica gel chromatography (ethyl acetate/methanol/triethylamine 85:10:5) to afford the title compound (1.35 g) as a yellow solid. ¹H NMR (300 MHz, DMSO_{d6}) δ 2.25 (s, 3H), 3.12 (m, 4H), 3.32 (m, 4H), 3.84 (m, 6H), 4.99 (m, 2H), 7.22 (d, 1H), 7.25 (s, 1H), 7.45 (d, 1H), 7.66 (d, 1H), 7.79 (d, 1H), 7.90 (d, 1H). ¹⁹F NMR (282 MHz, DMSO_{d6}) δ −115.0. MS (API-ES, M+H) 487.2. M. pt. 300° C. dec.

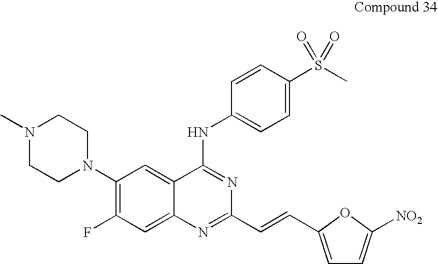

(E)-7-fluoro-6-(4-methylpiperazin-1-yl)-N-(4-(methylsulfonyl)phenyl)-2-(2-(5-nitrofuran-2-yl)vinyl)quinazolin-4-amine: This compound was prepared in the same manner as described for compound 30 but using 4-(methylsulfonyl)aniline instead of 4-aminophenol in the final step. ¹H NMR (300 MHz, DMSO_{d6}) δ 3.29 (s, 3H), 3.48 (s, 3H), 3.64 (m, 2H), 3.79 (m, 2H), 4.01 (m, 4H), 7.19 (d, 1H), 7.50 (d, 1H), 7.62 (d, 1H), 7.83, (d, 1H), 8.08 (d, 1H), 8.25 (m, 2H), 8.34 (m, 3H). MS (API-ES, M+H) 553.2. M. pt. 300° C. dec.

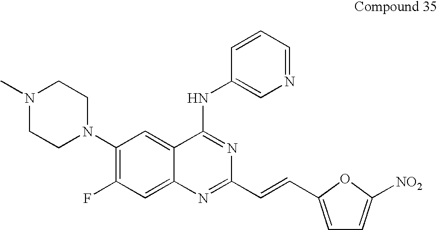

(E)-7-fluoro-6-(4-methylpiperazin-1-yl)-2-(2-(5-nitrofuran-2-yl)vinyl)-N-(pyridin-3-yl)quinazolin-4-amine: his compound was prepared in the same manner as described for compound 30 but using 3-aminopyridine instead of 4-aminophenol in the final step. ¹H NMR (300 MHz, DMSO_{d6}) δ 3.28 (s, 3H), 3.62 (m, 4H), 3.99 (m, 4H), 7.17 (m, 1H), 7.53 (d, 1H), 7.62 (m, 1H), 7.86 (d, 1H), 8.00 (s, 1H), 8.04 (d, 1H), 8.26 (m, 1H), 8.39 (m, 1H), 8.93 (m, 1H), 9.25 (m, 1H). MS (API-ES, M+H) 476.2. M. pt. 320° C. dec.

### EXAMPLE XVII

### Compounds 36 to 38

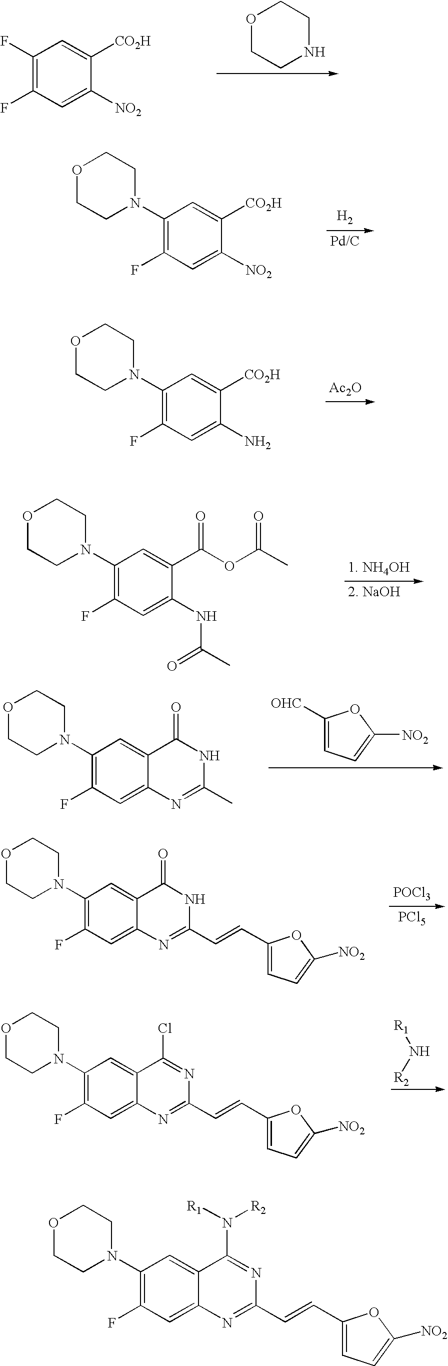

4-Fluoro-5-morpholin-4-yl-2-nitro-benzoic acid: A mixture of 2-nitro-4,5-difluorobenzoic acid (18.0 g, 88.626 mmol) and morpholine (400 ml) was heated at 80° C. for 3 h. After cooling, the mixture was concentrated in vacuo and dried under vacuum (60° C.) to provide the morpholine salt of the title compound (39.0 g) as a yellow solid. MS (M+H) 271.1.

2-Amino-4-fluoro-5-morpholin-4-yl-benzoic acid: A mixture of 4-Fluoro-5-morpholin-4-yl-2-nitro-benzoic acid (39.0 g) prepared above and 10% Pd/C (50% wet, 5 g) in ethanol (1.0 L) was subjected to hydrogenation at ambient temperature and 30 psi for 3 h. The mixture was filtered through Celite™, washed by ethanol and water, and the filtrate was concentrated in vacuo to afford the title compound (36.0 g) as an off-white solid. This compound was still the morpholine salt. MS (M+H) 241.1.

2-acetamido-4-fluoro-5-morpholinobenzoic acetic anhydride: A suspension of 2-Amino-4-fluoro-5-morpholin-4-yl-benzoic acid prepared above (36.0 g) in acetic anhydride (1.0 L) was refluxed overnight and then acetic anhydride was evaporated under vacuum in a rotavap to give an off-white solid.

7-Fluoro-2-methyl-6-morpholin-4-yl-3H-quinazolin-4-one: To the solid 2-acetamido-4-fluoro-5-morpholinobenzoic acetic anhydride was slowly added ammonium hydroxide solution (500 ml) under cooling with an ice bath and the resulting mixture was stirred overnight at ambient temperature. To the reaction mixture thus formed was added 10 wt % sodium hydroxide (400 ml). After refluxing for 1 hour, the reaction mixture was allowed to cool to ambient temperature and adjusted to pH 8 with conc. hydrochloric acid. The precipitate was filtered and washed with water, chased with toluene and dried under vacuum at 60° C. overnight to give the title compound (19.8 g, 85% overall yield for 3 steps) as an off-white solid. MS (M+H) 283.1.

7-Fluoro-6-morpholin-4-yl-2-[2-(5-nitro-furan-2-yl)-vinyl]-3H-quinazolin-4-one: To a solution of 7-Fluoro-2-methyl-6-morpholin-4-yl-3H-quinazolin-4-one (19.8 g, 75.2 mmol) in acetic acid (400 ml) was added 5-nitrofuraldehyde (15.91 g, 112.8 mmol) and conc. sulfuric acid (0.5 ml). After the reaction mixture was refluxed for one day, it was cooled to ambient temperature and concentrated in vacuo. The residue was triturated with EtOAc and dried under vacuum (50° C.) to afford the title compound (30.0 g, 99% yield) as a brown solid. MS (M+H) 283.1.

4-Chloro-7-fluoro-6-morpholin-4-yl-2-[2-(5-nitrofuran-2-yl)-vinyl]-quinazoline: To a suspension of 7-Fluoro-6-morpholin-4-yl-2-[2-(5-nitro-furan-2-yl)-vinyl]-3H-quinazolin-4-one (30.0 g, ˜75.2 mmol) in phosphorus oxychloride (400 ml) was added phosphorus pentachloride (29.5 g). After refluxing overnight, the reaction mixture was cooled, concentrated in vacuo, and diluted with ethyl ether. The precipitate was collected, washed with ethyl ether and dried under vacuum to give the title compound (30.5 g) as a dark-brown solid. MS (M+H) 405.0. ¹H NMR (300 MHz, DSMOd₆) δ 3.09 (m), 3.79 (m), 7.11 (d), 7.26 (d), 7.45 (d), 7.60 (d), 7.79 (m). ¹⁹F NMR (282 MHz, DSMOd₆) δ −113.0.

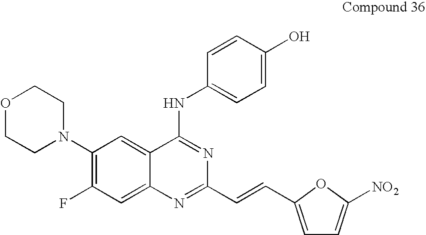

4-{7-Fluoro-6-morpholin-4-yl-2-[2-(5-nitro-furan-2-yl)-vinyl]-quinazolin-4-ylamino}-phenol: A mixture of 4-Chloro-7-fluoro-6-morpholin-4-yl-2-[2-(5-nitro-furan-2-yl)-vinyl]-quinazoline (6.07 g, 15 mmol) and 4-aminophenol (4.09 g, 37.5 mmol) in NMP (30 ml) was heated at 90° C. for 3 h. After cooling to ambient temperature, the mixture was poured into water (200 ml) and the precipitate was filtered, washed with water, methanol, and dried under vacuum at 50° C. to afford the title compound as a dark-brown solid. ¹H NMR (300 MHz, DSMOd₆) δ 3.19 (m, 4H), 3.82 (m, 4H), 6.85 (d, 2H), 7.21 (d, 1H), 7.27 (d, 1H), 7.56 (m, 4H), 7.78 (d, 1H), 7.93 (d, 1H), 9.63 (s, 1H). ¹⁹F NMR (282 MHz, DMSO_{d6}) δ −114.5. MS (API-ES, M+H) 487.2. M. pt. 176° C. dec.

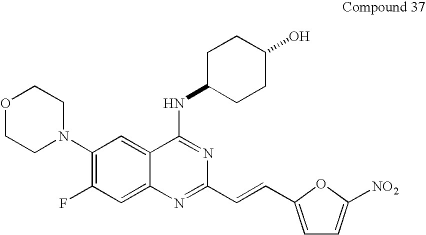

(E)-4-(7-fluoro-6-morpholino-2-((E)-2-(5-nitrofuran-2-yl)vinyl)quinazolin-4-ylamino)cyclohexanol: This compound was prepared in the same manner as compound 36 but using trans 4-amino-cyclohexanol instead of 4-aminophenol. ¹H NMR (300 MHz, DMSO_{d6}) δ 1.47 (m, 4H), 2.01 (m, H), 3.17 (m, 4H), 3.53 (m, 1H), 3.86 (m, 4H), 4.29 (m, 1H), 7.22 (d, 1H), 7.41 (d, 1H), 7.64 (d, 1H), 7.77 (d, 1H), 8.00 (d, 1H), 8.84 (d, 1H). MS (API-ES, M+H) 484.2. M. pt. 300° C. dec.

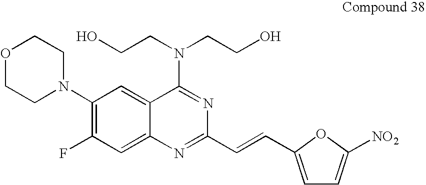

(E)-2,2′-(7-fluoro-6-morpholino-2-(2-(5-nitrofuran-2-yl)vinyl)quinazolin-4-ylazanediyl)diethanol: This compound was prepared from 4-Chloro-7-fluoro-6-morpholin-4-yl-2-[2-(5-nitro-furan-2-yl)-vinyl]-quinazoline and Bis-(2-trimothylsilanyloxy-ethyl)-amine as described for compound 4. ¹H NMR (300 MHz, DMSO_{d6}) δ 3.18 (m, 4H), 3.36 (m, 4H), 3.79 (m, 4H), 3.84 (m, 4H), 5.00 (bs, 1H), 7.26 (m, 2H), 7.48 d, 1H), 7.67 (d, 1H), 7.78 (m, 1H), 7.93 (m, 1H). ¹⁹F NMR (282 MHz, DMSO_{d6}) δ −115.1. MS (API-ES, M+H) 474.2. M. pt. 250° C. dec.

### EXAMPLE XVIII

### Compound 39

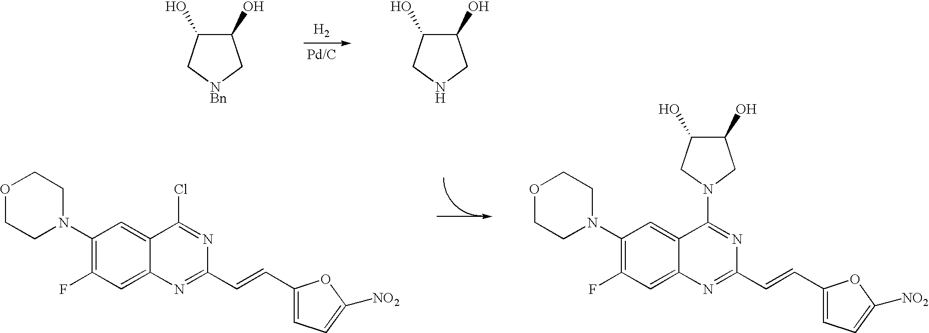

Trans-pyrrolidine-3,4-diol: A mixture of trans-1-benzylpyrrolidine-3,4-diol (5 g) and 10% Pd/C (50% wet, 1.0 g) in methanol (200 ml) was subjected to hydrogenation at ambient temperature at 50 psi overnight. The mixture was filtered through Celite™ and washed with methanol. The filtrate was concentrated in vacuo to afford the title compound (2.6 g) as a white solid.

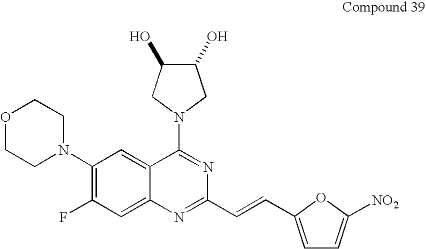

Trans-1-(7-fluoro-6-morpholino-2-((E)-2-(5-nitrofuran-2-yl)vinyl)quinazolin-4-yl)pyrrolidine-3,4-diol: A mixture of 4-Chloro-7-fluoro-6-morpholin-4-yl-2-[2-(5-nitro-furan-2-yl)-vinyl]-quinazoline (3.0 g) and trans-pyrrolidine-3,4-diol (2.6 g) in DMF (50 ml) was heated at 90° C. for 3 h. The mixture was concentrated under reduced pressure to remove most of DMF, and the residue was diluted with water and filtered to give the crude product as a black solid. This solid was subjected to flash chromatography (silica gel, eluted with MeOH/EtOAc 0-10% gradient) to afford the title compound (0.45 g) as an orange solid. ¹H NMR (300 MHz, DMSO_{d6}) δ 3.21 (m, 5H), 3.64 (m, 1H), 3.83 (m, 4H), 4.06 (m, 2H), 4.23 (m, 2), 7.26 (d, 1H), 7.34 (d, 1H), 7.80 (m, 3H), 8.08 (d, 1H). ¹⁹F NMR (282 MHz, DMSO_{d6}) δ −109.2. MS (API-ES, M+H) 472.2. M. pt. 250° C. dec.

### EXAMPLE XIX

### Compounds 40 to 44

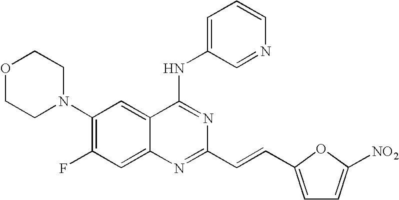

(E)-7-fluoro-6-morpholino-2-(2-(5-nitrofuran-2-yl)vinyl)-N-(pyridin-3-yl)quinazolin-4-amine: This compound was prepared in the same manner as compound 36 but using trans 3-aminopyridine instead of 4-aminophenol. ¹H NMR (300 MHz, DMSO_{d6}) δ 3.18 (m, 4H), 3.82 (m, 4H), 7.09 (m, 1H), 7.21 (m, 1H), 7.40 (m, 2H), 7.59 (m, 1H), 7.78 (m, 4H), 8.40 (m, 1H). MS (API-ES, M+H) 463.1. M. pt. 250° C. dec.

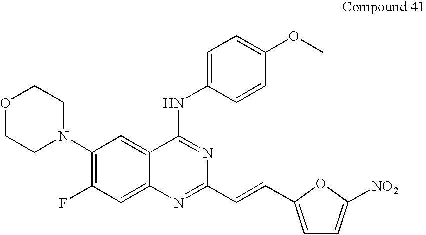

(E)-7-fluoro-N-(4-methoxyphenyl)-6-morpholino-2-(2-(5-nitrofuran-2-yl)vinyl)quinazolin-4-amine: This compound was prepared in the same manner as compound 36 but using trans 4-methoxy-aniline instead of 4-aminophenol. ¹H NMR (300 MHz, DMSO_{d6}) δ 3.18 (m, 4H), 3.80 (m, 4H), 7.04 (m, 1H), 7.17 (d, 1H), 7.28 (m, 1H), 7.56 (m, 2H), 7.66 (m, 2H), 7.76 (m, 2H), 7.97 (m, 1H), 10.19 (bs, 1H). MS (API-ES, M+H) 492.0. M. pt. 300° C. dec.

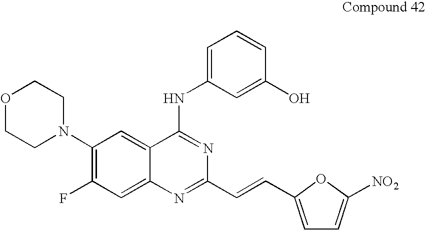

(E)-3-(7-fluoro-6-morpholino-2-(2-(5-nitrofuran-2-yl)vinyl)quinazolin-4-ylamino)phenol: This compound was prepared in the same manner as compound 36 but using trans 3-aminophenol instead of 4-aminophenol. ¹H NMR (300 MHz, DMSO_{d6}) δ 3.15 (m, 4H), 3.79 (m, 4H), 6.61 (m, 1H), 7.16-7.33 (m, 5H), 7.50 (m, 2H), 7.74 (m, 1H), 7.94 (d, 1H), 10.16 (bs, 1H). MS (API-ES, M+H) 478.2. M. pt. 300° C. dec.

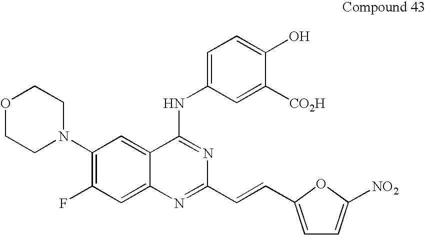

(E)-5-(7-fluoro-6-morpholino-2-(2-(5-nitrofuran-2-yl)vinyl)quinazolin-4-ylamino)-2-hydroxybenzoic acid: This compound was prepared in the same manner as compound 36 but using 5-amino-2-hydroxybenzoic acid instead of 4-aminophenol. ¹H NMR (300 MHz, DMSO_{d6}) δ 3.16 (m, 4H), 3.81 (m, 4H), 7.02 (m, 2H), 7.16 (m, 2H), 7.41 (m, 1H), 7.50 (m, 1H), 7.75 (m, 1H), 7.86 (m, 1H), 8.53 (s, 1H), 10.00 (bs, 1H). MS (API-ES, M+H) 522.2. M. pt. 300° C. dec.

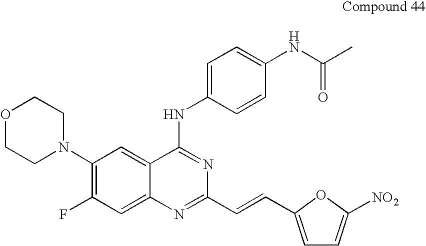

(E)-N-(4-(7-fluoro-6-morpholino-2-(2-(5-nitrofuran-2-yl)vinyl)quinazolin-4-ylamino)phenyl)acetamide: This compound was prepared in the same manner as compound 36 but using trans 4-acetamido-aniline instead of 4-aminophenol. ¹H NMR (300 MHz, DMSO_{d6}) δ 2.12 (s, 3H), 3.19 (m, 4H), 3.85 (m, 4H), 7.21 (m, 2H), 7.48 (m, 2H), 7.70 (m, 3H), 10.07 (bs, 2H). MS (API-ES, M+H) 519.2. M. pt. 300° C. dec.

### Methods of Compound Evaluation

Minimal Inhibitory Concentration (MIC) Determination. Bacteria (primary strain panel, TABLE 1). Susceptibility tests were performed following the recommendations from the National Committee for Clinical Standards (NCCLS). The MICs were determined by a broth microdilution technique using a final volume of 100 μl of cation-adjusted Mueller Hinton Broth (MHBCA) and a bacterial inoculum of 10⁵-10⁶ Colony Forming Units (CFU)/ml. The inocula were verified and precisely determined by applying 10 μl drops of 10-fold dilutions onto Triptic Soy Agar plates. The CFU were counted after an incubation of 24 h at 35° C. Any experiment showing an inoculum that was more or less than 10⁵-10⁶ CFU/ml was rejected. Control antibiotics and test compounds were prepared at a concentration equivalent to 2-fold the highest desired final concentration. Compounds were then diluted directly in the 96-well microtiter plates by serial 2-fold dilutions using a multichannel pipette. Microtiter plates were incubated during 24 h at 35° C. and growth was recorded by using a microtiterplate reader at 650 nm as well as by visual observation. The MIC was defined as the lowest concentration of compound yielding no visible growth. At least two commercial antibiotics (e.g., imipenem, ciprofloxacin, norfloxacin, nitrofurantoin, rifampicin, chloramphenicol, ampicillin, cefotaxime and vancomycin) were always included as internal microtiter plate controls in each MIC assay. Results from any microtiter plate that showed a discrepancy in such control antibiotic MICs compared to the NCCLS reference data for ATCC strains (a MIC differing by more than 2 doubling dilutions) were rejected. Fastidious bacteria. The medium used for Listeria monocytogenes, Neisseria meningitidis, and Campylobacter jejuni was MHBCA containing 2% laked horse blood. The medium used for Haemophilus influenzae and Branhamella (Moraxella) catarrhalis was HTM as recommended by the NCCLS. Cultures of these fastidious bacteria were incubated at 35° C. in a 5% CO₂ atmosphere. The MHBCA medium used to grow M. smegmatis prior to the MIC assays was supplemented with 0.02% Tween™-80 and results from microtiter plates were read after 48 hours of incubation. The medium used for Bacteroides fragilis was Wilkins Chalgren broth and growth was allowed under an anaerobic atmosphere at 35° C. for 48 hours.

Finally, compounds were also tested against populations of various clinical strains (antibiotic resistant strain panel, TABLE 1).

TABLE 1
Strain panels used in the evaluation of
antimicrobial activity of compounds.

```
Primary Strain Panel:
Gram positive.
Staphylococcus aureus ATCC 29213
Staphylococcus aureus MRSA COL
Staphylococcus epidermidis ATCC 12228
Staphylococcus saprophyticus ATCC 15305
Enterococcus faecalis ATCC 29212
Enterococcus faecium ATCC 35667
Bacillus cereus ATCC 11778
Bacillus subtilis ATCC 6633
Bacillus atrophaeus ATCC 9372
Listeria monocytogenes* ATCC 13932
Gram negative.
Escherichia coli ATCC 25922
Escherichia coli MC4100
Salmonella typhimurium ATCC 14028
Acinetobacter baumannii ATCC 19606
Yersinia enterocolytica ATCC 23715
Haemophilus influenzae* ATCC 49247
Haemophilus influenzae* ATCC 49766
Branhamella (Moraxella) catarrhalis* ATCC 8176
Campylobacter jejuni* ATCC 33291
Anaerobic bacteria.
Bacteroides fragilis* ATCC 25285
Acid-Fast bacteria.
Mycobacterium smegmatis* ATCC 19420
Antibiotic Resistant Strain Panel:
10 Staphylococcus aureus MRSA
  8 Escherichia coli
  1 Enterococcus faecium VRE (vanA)
Note:
*= Fastidious bacterial species
```

Minimal Bactericidal Concentration (MBC). After the microtiter plates were read for the determination of the MIC, a 10 μl sample of each clear well (at least 5 wells without visible growth) was applied onto TSA plates for viable counts determination.

Petri dishes were incubated at 35° C. for exactly 24 h and bacterial colonies were counted. The MBC was the minimal concentration of antibiotic which resulted in 99.9% killing of the original inoculum. For example, if the original inoculum was 1×10⁶ CFU/ml, the MBC was the concentration showing ≦10 colonies on TSA plate.

Time-kill curves. The bactericidal action of compounds was also evaluated over time (time-kill curve experiments). A bacterial inoculum of 1×10⁵-1×10⁷ Colony Forming Units (CFU)/ml was prepared. The inocula were verified and precisely determined by applying 10 μl drops of 10-fold dilutions onto Triptic Soy Agar plates. The CFU were counted after an incubation of 24 h at 35° C. Any experiment showing an inoculum that was more or less than the desired range of CFU/ml was rejected. Time-kill curve experiments were performed in 30 ml of MHB placed in 50 ml shaking flasks over a period of 24 hours. Test compounds and control antibiotics were added at time 0 hour and, at each time point, a sample was removed from flasks and the CFU determined by plate counts as described above. CFU from compound-treated cultures were compared to CFU collected from the control flask without antibiotic. Test compounds and control antibiotics were assayed at the MIC or a multiple of the MIC as determined by a broth microdilution technique as described above.

In vivo efficacy. A thigh infection model in the neutropenic mouse was used as the experimental model. Mice were challenged with Staphylococcus aureus ATCC 29213 administered i.m. in 0.1 ml volume to each thigh. To determine efficacy, compounds were delivered i.v. in a single dose 2 h post infection. Mice (3 mice or more per treatment) were euthanized 8 h post infection. The thigh tissue (two samples per animal) was recovered, homogenized, and CFU/g tissue determined by plating appropriate dilutions. Statistical analysis was done by unpaired Student's t-test using the GraphPad Prism™ software. Values of p≦0.05 were considered to be significant.

### Results of Compound Evaluation

Inhibitory activity of Example I (compound V) and Example VII (compound XV). The compounds from Examples I and VII were evaluated against panels of microorganisms as described in TABLE 1 in order to determine their relative potency (MICs and MBCs) and breadth of spectrum. In the results outlined in TABLE 2, many reference microorganisms (American Type Culture Collection, ATCC strains) and many commercially available antibiotics were included in each of the tests used to characterize the activity of Examples I and VII in order to validate measurements and ensure high quality data.

TABLE 2
MICs (and MBCs) in μg/ml for control antibiotics and compounds of Examples I and
VII obtained for a variety of Gram positive bacteria from the primary strain panel.

| | S. aureus | S. aureus | S. epidermidis ATCC | S. saprophyticus ATCC | | E. faecalis | E. faecium |
|---|---|---|---|---|---|---|---|
| Antibiotic | ATCC 29213 | MRSA COL | 12228 | 15305 | | ATCC 29212 | ATCC 35667 |
| Example I | 0.03-0.06 (0.06) | 0.015 (0.06) | 0.03 (0.03) | 0.015-0.03 | (0.03-0.06) | 0.03 (0.03) | 0.06 (0.25) |
| compound V | | | | | | | |
| Example VII | 2 | 0.5 | 1-2 | 2 | | 2-8 | 8 |
| compound XV | | | | | | | |
| Ampicillin | 1-2 | 8 | 64 | 0.12 | | 1 | 1 |
| Cefotaxime | 1-2 | 512 | 0.5-1 | 8 | | 2-8 | 16 |
| Ceftriaxone | 2 | 512 | 1 | 8 | | 8 | 64 |
| Chloramphenicol | 16 | 8 | 8 | 4-8 | (16-32) | 8 | 8 |
| Erythromycin | 0.5 | 0.25 | 0.5 | 0.25 | | 2-4 | 2 |
| Furazolidone | 4-16 | 8 | 2-4 | 2 | | 8-16 | >128 |
| Gentamicin | 0.5-2 | 0.5 | 0.06-0.25 | 0.06 | | 4-16 | 4-16 |
| Imipenem | 0.015 | 16 | 0.008-0.015 | 0.03 | (0.03-0.06) | 0.5 | 2 |
| Meropenem | 0.06 | 16 | 0.06 | 0.25 | | 2-4 | 16 |
| Nitrofurantoin | 16 | 16 | 16 | 8 | (16) | 8-16 | 64 |
| Nitrofurazone | 16 | 8-16 | 8 | 8 | | 64 | 128 |
| Norfloxacin | 1 | 0.5-1 | 0.5 | 2 | (4) | 4 | 16 |
| Oxacillin | 0.12-0.25 | 512 | 0.12 | 1 | | 8-16 | 32 |
| Rifampicin | 0.008-0.015 | 0.008-0.015 | 0.004-0.008 | 0.03 | | 0.5-1 | 32 |
| Tetracycline | 0.5-1 | 2 | 128 | 1 | | 16-32 | 0.5 |
| TMP/SMX (1/19) | 0.06/1.2 | 0.25/4.8-0.5/9.5 | 0.12/2.4-0.25/4.8 | 0.06/1.2 | (0.25/4.8) | 0.015/0.3 | 0.12/2.4 |
| Vancomycin | 0.5-2 | 1-2 | 1-2 | 1 | (1) | 2 | 0.5 |

Compound V and Compound XV showed unexpected exquisite activities against Gram positive bacteria which generally cause severe opportunistic and/or nosocomial infections (TABLE 2). These included Methicillin-Resistant and Methicillin-Sensitive S. aureus strains [MRSA and MSSA, respectively], S. epidermitidis, E. faecalis and E. faecium. The activity of Example I was unexpectedly better than that of imipenem, norfloxacin, vancomycin or several other commercial antibiotics against MRSA, E. faecalis and E. faecium. Compound XV was unexpectedly better than commercial nitrofurans like nitrofurantoin and nitrofurazone against all strains of TABLE 2.

Compound V was also very active against pathogens often causing urinary tract infections (e.g., S. saprophyticus, TABLE 2, and E. coli, TABLE 3). Against the reference strains, the activity of Compound V was unexpectedly better than commercial nitrofuran agents, like nitrofurantoin, usually used for treatment of urinary tract infections.

The MBCs of Compound V were most of the time equal to or only 2 to 4-fold higher than the MICs showing that this compound was bactericidal and not bacteriostatic.

Compound V also demonstrated an unexpected very good activity against three species of the bacterial genus Bacillus (i.e., B. cereus, B. subtilis and B. atrophaeus) with MICs ranging from 0.03 to 0.125 μg/ml (data not shown). Bacillus anthracis, the bacterial pathogen causing anthrax, is also a member of that bacterial genus.

Compounds V and XV showed excellent activity against respiratory tract pathogens causing community-acquired otitis media and pneumonia (TABLE 4). The activity of Compound V was unexpectedly superior to that of the β-lactam drugs (ampicillin, cefotaxime and meropenem) and macrolides (erythromycin, clarithromycin) against H. influenzae ATCC 49247 and B. catarrhalis. Compounds V and XV were also very active against Mycobacterium smegmatis and their activity was unexpectedly superior to that of the commercial nitrofurans, norfloxacin and rifampicin. Mycobacterium tuberculosis, the bacterial pathogen causing tuberculosis, is a member of that bacterial genus. Compound V was very active against L. monocytogenes and C. jejuni causing enteric infections and against B. fragilis, an anaerobe often causing difficult-to-treat abscesses and infections in diabetic patients.

TABLE 3
MICs (and MBCs) in μg/ml for control antibiotics and compound of Example I obtained
for a variety of Gram negative bacteria from the primary strain panel.

| | E. coli | S. typhimurium | A. baumannii | Y. enterocolytica |
|---|---|---|---|---|
| Compounds | ATCC 25922 | ATCC14028 | ATCC 19606 | ATCC 23715 |
| Example I compound V | 0.5 (0.5) | 1 | 1-2 | 0.25 |
| Ampicillin | 4-8 | — | — | — |
| Meropenem | 0.015-0.06 | — | 2 | >0.5 |
| Chloramphenicol | 4 (16) | — | — | — |
| Cefotaxime | 0.06-0.12 | >2 | 16 | >2 |
| Imipenem | 0.12 | — | 16 | 0.5 |
| Ceftriaxone | 0.03-0.06 | — | — | — |
| Oxacillin | 512->512 | — | — | — |
| Erythromycin | 64 | — | — | — |
| Rifampicin | 8 | — | 4 | 8 |
| Norfloxacin | 0.03 (0.06) | — | — | — |
| Tetracycline | 1-2 | — | 16 | >32 |
| Gentamicin | 0.5-2 | — | 32 | 2 |
| Nitrofurantoin | 8 (8) | 32 | 128 | 64 |
| Nitrofurazone | 8-16 | 8 | 32 | 64 |
| Furazolidone | 1-2 | 2 | 32 | >16 |
| TMP/SMX (1/19) | 0.25/4.75-0.5/9.5 | — | 8/152 | 0.125/2.4 |

TABLE 4
MICs (and MBCs) in μg/ml of control antibiotics and compounds of Examples I and VII
obtained for Gram negative, Gram positive and acid-fast fastidious bacterial species.

| | H. influenzae | | H. influenzae | B. catarrhalis | M. smegmatis |
|---|---|---|---|---|---|
| Compounds | ATCC 49247 | | ATCC 49766 | ATCC 8176 | ATCC 19420 |
| Example I compound V | 0.03 | (0.06) | 0.06 (0.06) | 0.008 (0.03) | 0.06 |
| Example VII compound XV | 2 | | 2 | 0.5 | 0.25 |
| Ampicillin | 2-4 | (4) | ≦0.12 (≦0.12) | ≦0.03 (≦0.03) | — |
| Cefotaxime | 0.12 | (0.12) | 0.008 (0.008) | 0.03 (0.06) | — |
| Ciprofloxacin | 0.008-.015 | (0.015) | 0.008 (0.015) | 0.03 (0.06) | — |
| Clarithromycin | 16 | | 128 (>128) | 0.5 (1) | — |
| Erythromycin | 2 | (2) | 8 (16) | 0.06 (0.12) | — |
| Meropenem | 0.06 | (0.06) | 0.03 (0.06) | 0.002 (0.008) | — |

| | | | | |
|---|---|---|---|---|
| Nitrofurantoin | — | — | — | 64 |
| Nitrofurazone | — | — | — | 64 |
| Norfloxacin | — | — | — | 2 |
| Rifampicin | — | — | — | 8 |

| | L. monocytogenes | C. jejuni | B. fragilis |
|---|---|---|---|
| Compounds | ATCC 13932 | ATCC 33291 | ATCC 25285 |
| Example I compound V | 0.03-0.25 | 0.016 | 0.016 |
| Example VII compound XV | 0.5 | — | 0.5 |
| Chloramphenicol | 0.001 | 4 | 4 |
| Rifampicin | 0.06 | — | — |
| Norfloxacin | 0.008 | — | — |
| Tetracycline | — | 0.5 | 0.25 |
| Imipenem | — | 0.125 | 0.125-0.5 |

The activity of Compounds V and XV was not influenced by the resistance mechanisms residing in multi-resistant E. faecium (e.g., strain VanA, TABLE 5). Similarly, the activity of Compounds V and XV was not influenced by the resistance mechanisms residing in multi-resistant MRSA strains (TABLE 6). This data was unexpected considering that at least 80% of the strains that were tested were resistant to many antibiotics of the conventional arsenal (e.g., oxacillin, erythromycin, norfloxacin).

The activity of Compound V was also not influenced by the resistance mechanisms residing in multi-resistant E. coli (e.g., strains Ec022c, Ec027c, Ec117c, Ec118c, and Ec119c, TABLE 7) or by the pathotype, i.e., the virulence characteristics of the strains (e.g., Entero-Hemorrhagic E. coli O157:H7 or Extra-Intestinal E. coli EIEC, TABLE 7).

The activity of Compounds V and XV against multi-resistant microorganisms, i.e., that are resistant to at least two structural classes of drugs, indicates that the chemical nature of the nitrofurans of the present invention was not previously encountered by such strains or did not elicit the development of resistance among these strains as opposed to all the other antibiotic classes that were tested (TABLES 5, 6, and 7).

TABLE 5
MICs in μg/ml of control antibiotics and compounds
of Examples I and VII obtained for antibiotic multi-resistant
Enterococcus faecium (VanA).

| | E. faecalis | E. faecium | E. faecium |
|---|---|---|---|
| Compounds | ATCC 29212 | ATCC 35667 | VanA |
| Example I compound V | 0.03 | 0.06 | 0.03 |
| Example VII compound | 2-8 | 8 | 0.5 |
| XV | | | |
| Vancomycin | 2 | 0.5 | >128 |
| Cefotaxime | 2-8 | 16 | >128 |
| Clarithromycin | 16 | — | >128 |
| Tetracycline | 16-32 | 0.5 | 128 |
| Rifampicin | 0.5-1 | 32 | >128 |
| Furazolidone | 8-16 | >64 | 64 |
| Nitrofurazone | 64 | >64 | 64 |

TABLE 6
MICs in μg/ml for control antibiotics and compounds of Examples I and
VII obtained for a variety of antibiotic multi-resistant MRSA strains.

| MRSA strains | Example I | Example VII | | | | | |
|---|---|---|---|---|---|---|---|
| (n = 10) | compound V | compound XV | Oxacillin | Erythromycin | Norfloxacin | Gentamicin | Nitrofurantoin |
| MRSA COL | 0.015 | 0.5 | >128 | 0.25 | 1 | 0.5 | 8-16 |
| Sa211c | ≦0.06 | 2 | 16-32 | >32 | >32 | 1 | 16-32 |
| Sa212c | 0.125 | 4 | 16 | >32 | >32 | 0.25 | 32 |
| Sa220c | ≦0.06 | 0.25 | 8-16 | 0.5 | >32 | 0.5 | 16 |
| Sa224c | ≦0.06 | 1 | 32-64 | >32 | >32 | 0.25 | 16 |
| Sa228c | ≦0.06 | 2 | 128 | >32 | >32 | 32 | 16-32 |
| Sa234c | ≦0.06 | 2 | 32->128 | >32 | >32 | 1 | 16-32 |
| Sa248c | ≦0.06 | 0.5 | 512 | >128 | 32 | >128 | 16 |
| Sa249c | ≦0.06 | 0.5 | 512 | >128 | 32 | >128 | 16 |
| Sa253c | ≦0.06 | 2 | 1024 | >128 | >128 | >128 | 16 |

TABLE 7
MICs in μg/ml for control antibiotics and compound of Example I obtained for a
variety of antibiotic resistant E. coli strains and/or of different pathotypes.

| E. coli | | Example I | | | | TMP-SMX | |
|---|---|---|---|---|---|---|---|
| strains (n = 10) | Pathotype | compound V | Ampicillin | Ciprofloxacin | Nitrofurantoin | (1/19) | Tetracycline |
| ATCC 25922 | Reference | 0.5 | 4 | 0.03 | 8-16 | 0.25/4.75-0.5/9.5 | 2 |
| MC4100 | Reference | 1 | 4 | — | 8-16 | — | — |
| ATCC 35150 | O157: H7 | 1 | 2 | ≦0.25 | 2 | 0.03/0.6 | 2 |
| d4-OLR-pen10 | O157: H7 | 1 | 2-4 | ≦0.25 | 16 | 0.125/2.4 | >32 |
| d25-ALR-pen14 | O157: H7 | 1 | 2-4 | ≦0.25 | 16 | 0.125/2.4 | >32 |
| Ec022c | EIEC | 1 | >64 | 0.015-0.03 | 16 | >2/38 | 2 |
| Ec027c | EIEC | 1 | >64 | 0.015 | 16 | 0.03/0.6 | >32 |
| Ec117c | EIEC | 1 | 16 | >128 | 8-16 | >2/38 | >32 |
| Ec118c | EIEC | 1 | >64 | 64 | 16 | >2/38 | 4 |
| Ec119c | EIEC | 2 | >64 | 32 | 16 | >2/38 | >32 |

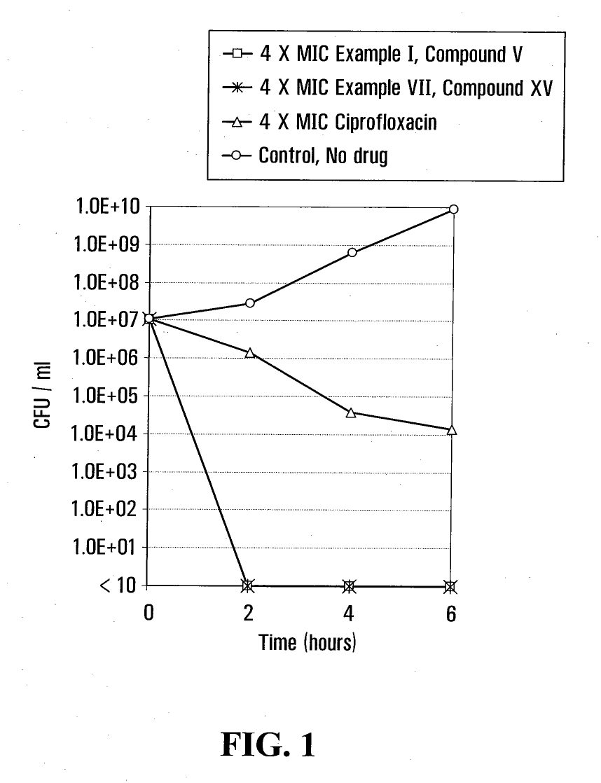

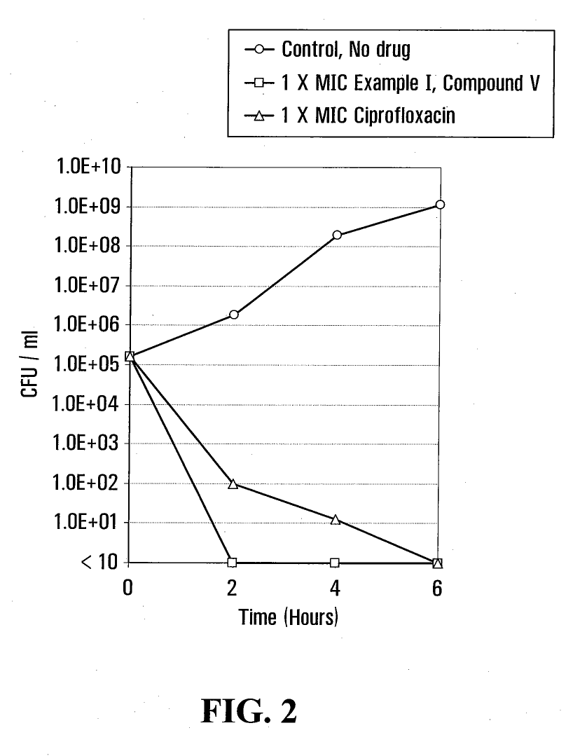

Bactericidal activity of Compounds V and XV. Compound V of Example I and compound XV of Example VII were evaluated in time-kill studies against S. aureus and/or E. coli (FIGS. 1 and 2, respectively). Results showed that Compounds V and VII were unexpectedly bactericidal within 2 hours against the tested strains. Compound V was unexpectedly superior to ciprofloxacin at their respective MIC or a multiple of the MIC against both species. Compound V was similarly bactericidal against strain S. aureus MRSA COL showing that it is also able to kill bacteria resistant to commonly used antibiotics (data not shown). Accordingly, the person skilled in the art would predict that the compounds of the invention would behave in a similar way and show substantially similar utility.

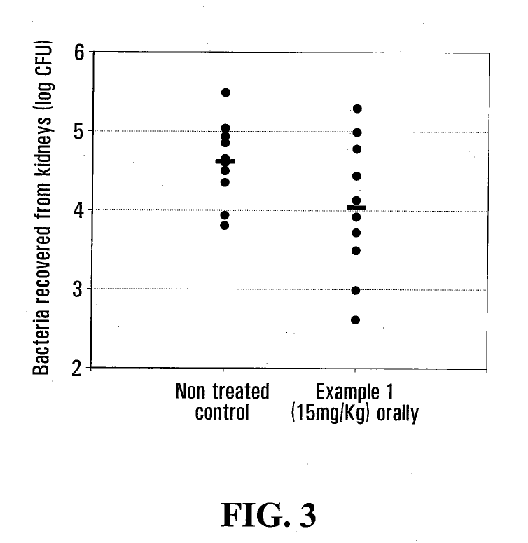

In vivo activity of Compound V. Compound V was active in vivo. FIG. 3 reports the results of a S. aureus peritonitis model of infection in the mouse. Results clearly showed that Compound V reduced significantly the presence of viable bacteria in the kidneys. This result demonstrated oral bioavailability of Compound V and its relatively low toxicity in vivo. Accordingly, the person skilled in the art would predict that the compounds of the invention would behave in a similar way and show substantially similar utility.

Solubility of Compounds V and XV. The extent of solubility of compounds was evaluated in water. Compound V was soluble in water (no visible particles) at a concentration of 0.25 mg/ml, whereas Compound XV was 4 times more soluble (i.e., 1 mg/ml).

Bactericidal activity of Compounds 1 to 44. As described for compounds of Examples I and VII above, compounds 1 to 44 of Examples VIII to XIX were also evaluated to obtain their relative potency and breath of spectrum against a representative panel of reference organisms (see TABLE 8).

TABLE 8
IN VITRO EVALUATION OF COMPOUNDS 1 TO 44. THE TABLE DEPICTS THE MINIMUM INHIBITORY
CONCENTRATIONS TO KILL THE BACTERIA IN MICROGRAMS PER MILLILITER.

| | | | | Esch- | | | | | |
|---|---|---|---|---|---|---|---|---|---|
| | | | | erichia | | | | | |
| | Staphylococcus | Staphylococcus | Staphylococcus | coli | Enterococcus | Enterococcus | Pseudomonas | Streptococcus | Haemophilus |
| Com- | aureus | epidermidis | saprophyticus | ATCC | faecium | faecalis | aeruginosa | pneumoniae | influenzae |
| pound | ATCC 29213 | ATCC 12228 | ATCC 15305 | 25922 | ATCC 35667 | ATCC 29212 | ATCC 27853 | ATCC 49619 | ATCC49766 |
| 1 | 0.03 | 0.03 | 0.004 | 0.25 | 0.03 | 0.008 | >64 | 0.008 | 0.015 |
| 2 | 2 | 0.5 | 0.25 | 8 | 0.25 | 0.12 | >64 | 0.12 | 2 |
| 3 | 0.03-0.06 | 0.06 | 0.015-0.06 | 16->64 | 0.12-1 | 0.03-0.06 | >64 | 0.008-0.03 | 0.06 |
| 4 | 0.5 | 0.12 | 0.06 | 2 | 0.25 | 0.06 | 64 | 0.03 | 0.5 |
| 5 | 2 | 0.25 | 0.06 | >64 | 4 | 0.25 | >64 | 0.03 | 0.5 |
| 6 | 2 | 1 | 1 | 4 | 1 | 2 | >64 | 0.06 | 4 |
| 7 | 1 | 0.25 | 0.25 | 4 | 0.5 | 1 | >64 | 0.06 | 2 |
| 8 | 4 | 0.12 | 0.06 | >64 | 0.5 | 0.06 | >64 | 0.03 | 0.5 |
| 9 | 0.5 | 0.25 | 0.25 | >64 | 1 | 0.25 | >64 | 0.12 | 1 |
| 10 | 1 | 0.5 | 0.25 | >64 | 1 | 0.5 | >64 | 0.25 | 4 |
| 11 | 0.5 | 0.25 | 0.5 | 8 | 0.5 | 0.25 | >64 | 0.008 | 1 |
| 12 | 0.25 | 0.03 | 0.06 | >64 | 2 | 0.12 | >64 | 0.008 | 0.5 |
| 13 | N/A | N/A | N/A | N/A | N/A | N/A | N/A | N/A | N/A |
| 14 | 0.25 | 0.12 | — | >64 | 1 | 0.12 | >64 | 0.015 | 0.25 |
| 15 | 0.03 | 0.015-0.06 | 0.008-0.015 | >64 | 0.06-0.12 | 0.008-0.03 | >64 | 0.015-0.06 | 0.03-0.06 |
| 16 | 2 | 1 | 1 | 16 | 0.25 | 0.12 | >64 | 0.25 | 8 |
| 17 | 0.06-0.25 | 0.12 | 0.03-0.06 | >64 | 0.12-0.25 | 0.06 | >64 | 0.015 | 0.5 |
| 18 | 0.25 | 0.12 | 0.12 | 2 | 0.5 | 0.25 | >64 | N/A | N/A |
| 19 | 0.5 | 0.25 | 0.12 | 4 | 1 | 0.5 | >64 | N/A | N/A |
| 20 | 0.25 | 0.06 | 0.12 | 2 | 0.25-0.5 | 0.12 | >64 | 0.008-0.015 | 0.5 |
| 21 | 0.015 | 0.008 | 0.008-0.015 | 0.5-1 | 0.06-0.12 | 0.015-0.03 | >64 | 0.008-0.015 | 0.06 |
| 22 | 0.03 | 0.03 | 0.06 | 2 | 0.125 | 0.25 | >16 | 0.015 | 0.125 |
| 23 | >16 | >16 | >16 | >16 | >2 | >2 | >16 | >2 | >16 |
| 24 | 0.5 | 0.25 | 0.25 | >16 | 1 | 0.125 | >16 | 0.125 | 2 |
| 25 | 0.06 | 0.008-0.03 | 0.015-0.03 | >64 | 0.12 | 0.03-0.06 | >64 | N/A | 0.06 |
| 26 | 0.5 | 0.12 | 0.12 | >64 | 0.25 | 0.12 | >64 | N/A | N/A |
| 27 | 0.12 | 0.12 | 0.06 | >64 | 0.12 | 0.12 | >64 | N/A | N/A |
| 28 | >2 | 2 | 2 | >64 | 2 | 1 | >64 | N/A | N/A |
| 29 | >2 | 2 | 2 | >16 | >2 | 1 | >16 | 1 | >2 |
| 30 | 0.015-0.06 | 0.03 | 0.03-0.06 | 0.5 | 0.12-0.25 | 0.03-0.06 | >64 | 0.008 | 0.25 |
| 31 | 1 | 0.25 | 0.25 | 4 | 1 | 0.12 | 8-16 | 0.015 | 1 |
| 32 | 1 | 0.5 | 0.5 | 8 | 4 | 0.5 | >64 | 0.06 | 2 |
| 33 | 1 | 0.25 | 0.5 | 2 | 1 | 0.5 | 8-16 | 0.03 | 4 |
| 34 | 0.25 | 0.06 | 0.06 | 8->64 | 2 | 0.06 | >64 | 0.004 | 0.5 |
| 35 | 0.5 | 0.25 | 0.5 | 1 | 1-2 | 0.5-1 | >64 | 0.015 | 2 |
| 36 | 0.03-0.06 | 0.015-0.06 | 0.015-0.06 | >64 | 0.12-0.25 | 0.015-0.06 | >64 | 0.008-0.03 | 0.12-0.5 |
| 37 | 0.06 | 0.06 | 0.03 | N/A | 0.5 | 0.25 | 64 | 0.015 | 0.25 |
| 38 | 0.25 | 0.25 | 0.25 | >2 | N/A | 0.25 | N/A | 0.03 | 2 |
| 39 | 0.06 | 0.06 | 0.12 | >2 | N/A | 0.12 | N/A | 0.015 | 1 |
| 40 | 0.03 | 0.015 | 0.03 | 2 | 0.25 | 0.03-0.06 | 64 | 0.008 | 0.12-0.25 |
| 41 | 0.25 | 0.06 | 0.06 | 64 | 0.5 | 0.25 | 64 | 0.06 | 0.5 |
| 42 | 0.06 | 0.06 | 0.03 | — | 0.5 | 0.25 | 64 | 0.015 | 0.25 |
| 43 | 2 | >2 | >2 | >64 | >64 | 2 | 64 | 0.5 | >2 |
| 44 | 0.06 | 0.03 | 0.03 | 64 | 0.25 | 0.06 | 64 | 0.008 | 0.25 |

The demonstrated in vitro activity in TABLE 8 clearly shows that the structural modifications of compounds 1 to 44 are associated with various degrees of activity.

Compounds 1 to 44 shown in TABLE 8 have minimum inhibitory concentrations against S. aureus, S. epidermidis, E. faecalis and E. Faecium with very good values within a range that is equivalent or better when compared to the common antibiotics found in TABLE 2. The person skilled in the art will readily understand that these results support the notion that a compound of the invention is useful to treat nosocomial as well as community acquired infections, which cause infections of the skin and tissue, as well as systemic infections such as for example, but which are not limited to, endocarditis.

Compounds 1 to 44 shown in TABLE 8 have minimum inhibitory concentrations against S. pneumoniae and H. influenzae with values within a range that is equivalent or better when compared to the common antibiotics found in TABLE 4. From these results, the person skilled in the art will readily understand that a compound of the invention is useful for treatment of, but which are not limited to, infections of the respiratory tract.

In a preferred embodiment, the compound of the invention does not have an acetate group on the nitrogen at the 4 position of quinazoline.

Compounds 1, 21, 22 and 30 show remarkable activity against S. aureus as well as activity against E. coli. While others, such as compounds 3, 15, 25 and 36, are highly potent against S. aureus, they lose E. coli activity. The person skilled in the art will readily understand that the loss of E. coli activity may be advantageous as, in some cases, it may be beneficial to preserve intestinal flora. Similarly, the person skilled in the art would be able to determine against which pathogen a given compound of the invention would have utility, without undue experimentation, e.g. with the herein disclosed data and methods, and with methods known in the art.

The results for selected compounds shown in TABLE 8 were compared to the MIC₉₀ data to assure that the values generated from the ATCC reference strains are illustrative of human clinical infections. The comparisons are shown in TABLE 9.

TABLE 9
COMPARISON OF MIC₉₀ WITH REFERENCE ORGANISM DATA FROM TABLE 8. THE TABLE
DEPICTS THE MINIMUM INHIBITORY CONCENTRATIONS IN MICROGRAMS PER MILLILITER
FOR WHICH 90 PERCENT OF CLINICAL STRAINS TESTED ARE GROWTH INHIBITED AND
COMPARES IT TO THE DATA ON THE REFERENCE STRAINS FROM TABLE 8.

| | | | | MIC₉₀ | | | | |
|---|---|---|---|---|---|---|---|---|
| | MIC | | MIC | Penicillin | | MIC₉₀ | MIC | MIC₉₀ |
| | Staphylococcus | MIC₉₀ | Streptococcus | Resistant | MIC | Clinical | Heamophilus | Clinical |
| | aureus | MRSA | pneumoniae | S. pneumoniae | feacalis | E. feacalis | influenzae | H. Influenzae |
| Compound | ATCC 29212 | (n = 23) | ATCC 49619 | (n = 15) | ATCC 29212 | (n = 17) | ATCC 49766 | (n = 23) |
| 15 | 0.03 | 0.03 | 0.015-0.006 | 0.015 | 0.008-0.03 | 0.06 | 0.03-0.06 | 0.125 |
| 30 | 0.015-0.06 | 0.03 | 0.008 | 0.03 | 0.03-0.06 | 0.125 | 0.25 | 0.25 |
| 36 | 0.03-0.06 | 0.03 | 0.008-0.03 | 0.03 | 0.015-0.006 | 0.125 | 0.125-0.5 | 0.25 |
| Vancomycin | 0.5-2 | 1 | — | — | 2 | 1 | — | — |
| Penicillin G | — | — | 0.25 | 2 | — | — | — | — |
| Erythromycin | — | — | 0.015 | >128 | 4 | >128 | 8 | 4 |

The comparisons of the MIC values for compounds 15, and 36 against representative strains to the MIC₉₀ data taken from clinical isolates of S. aureus, S. pneumoniae, E. feacalis and H. influenzae demonstrate that the MIC data is indeed representative. The person skilled in the art will thus understand that these results support the use of a compound of the invention in a method of treatment of human infections.

Furthermore, ten compounds from compounds 1 to 44 were tested in vivo in a mouse thigh infection model caused by S. aureus. The mice were administered selected compounds post infection by either intravenous (I.V.) or oral (P.O.) routes. The results are shown in TABLE 10.

Compounds 15, 17, 21, 36 and 40 showed 2 log reductions in bacterial load after I.V. administration. The person skilled in the art will readily understand that these data support the notion that a compound of the invention reaches the infection site and acts to help remove the bacteria in mammals. Accordingly, from the results in TABLE 10, the person skilled in the art would readily understand the notion that a compound of the invention may be used as an agent to treat bacterial infections in mammals, and preferably in humans.

TABLE 10
Results from In Vivo Efficacy in a Mouse Thigh Infection
Model Caused by Staphylococcus aureus ATCC 29213.

| | | | Average CFU (Log10) | | |
|---|---|---|---|---|---|
| | Route of | Dose | Non- | | Log |
| Compound | administration | (mg/kg) | Treated | Treated | difference |
| 7 | I.V. | 35 | 7.2 | 6.6 | 0.6 |
| | I.V. | 52 | 7.2 | 6.5 | 0.7 |
| | P.O. | 325 | 7.3 | 6.3 | 1.0 |
| 8 | I.V. | 16 | 6.8 | 6.7 | 0.1 |
| | I.V. | 2 × 12* | 6.8 | 6.7 | 0.1 |
| 15 | I.V. | 20 | 7.2 | 6.5 | 0.7 |
| | I.V. | 26 | 6.9 | 4.6 | 2.3 |
| | I.V. | 40 | 7.2 | 5.5 | 1.7 |
| 17 | I.V. | 16 | 7.3 | 5.6 | 1.7 |
| 20 | I.V. | 16 | 7.4 | 7.0 | 0.4 |
| 21 | I.V. | 4.5 | 7.6 | 7 | 0.6 |
| | I.V. | 9 | 7.6 | 6.3 | 1.3 |
| | I.V. | 12 | 7.4 | 5.2 | 2.2 |
| | I.V. | 14 | 7.3 | 4.6 | 2.7 |
| | I.V. | 18 | 7.6 | 4.6 | 3.0 |
| | P.O. | 208 | 7.0 | 6.8 | 0.2 |
| 22 | P.O. | 186 | 6.9 | 6.4 | 0.5 |
| 30 | I.V. | 17 | 7.7 | 7.1 | 0.6 |
| | I.V. | 26 | 7.7 | 7.0 | 0.7 |
| 36 | I.V. | 4.5 | 7.2 | 6.8 | 0.4 |
| | I.V. | 9 | 7.2 | 6.7 | 0.5 |
| | I.V. | 12 | 7.2 | 5.4 | 1.8 |
| | I.V. | 2 × 9* | 7.2 | 6.0 | 1.2 |
| | I.V. | 18 | 7.2 | 4.6 | 2.6 |
| | I.V. | 27 | 7.2 | 4.7 | 2.5 |
| | I.V. | 33 | 7.2 | 4.7 | 2.5 |
| | I.V. | 50 | 7.2 | 4.2 | 3.0 |
| | I.V. | 67 | 7.2 | 4.5 | 2.7 |
| 40 | I.V. | 14 | 7.4 | 4.8 | 2.6 |
| Linezolid | I.V. | 2.5 | 7.0 | 6.8 | 0.2 |
| | I.V. | 5 | 7.0 | 6.2 | 0.8 |
| | I.V. | 10 | 7.0 | 5.6 | 1.4 |
| | I.V. | 20 | 7.0 | 4.9 | 2.1 |
| Vancomycin | I.V. | 18 | 7.3 | 4.8 | 2.5 |
| | I.V. | 36 | 7.0 | 4.8 | 2.2 |
| CIPRO | P.O. | 186 | 6.9 | 4.9 | 2.0 |

*Double doses were given at T = 2 hr and 5 hr

Furthermore, as can be seen from the disclosed data, compounds 21 and 22, which are substantially identical except that in compound 21 X of formula 1.0 is trans CHCH, whereas in compound 22 X of formula 1.0 is absent, have substantially similar demonstrated activity. Accordingly, the person skilled in the art would reasonably predict that substantially similar compounds of the invention would have substantially similar activity having regard to X of formula 1.0 being absent, cis or trans CHCH.

While the invention has been described with reference to exemplary embodiments, those skilled in the art will understand that various changes may be made and equivalents may be substituted for elements thereof without departing from the scope of the invention. In addition, many modifications may be made to adapt a particular situation to the teachings of the invention without departing from the essential scope thereof. Therefore, it is intended that the invention not be limited to the particular embodiments for carrying out this invention, but that the invention will include all embodiments falling within the scope of the appended claims.

1. A compound of the formula 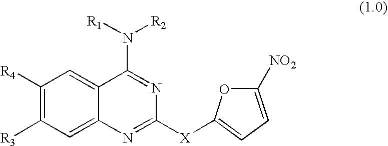
wherein:
X is absent, or trans or cis CHCH;
R₁ is (C₁-C₁₀)alkyl unsubstituted or substituted by one to three hydroxy, (C₂-C₁₀)alkenyl unsubstituted or substituted by one to three hydroxy, (C₂-C₁₀)alkynyl unsubstituted or substituted by one to three hydroxy, or aryl unsubstituted or substituted by one to three hydroxy;
R₂ is hydrogen, alkyl or aryl,
wherein R₁ and R₂ when taken together form a (C₂-C₁₀)cycloalkyl unsubstituted or substituted by one or two hydroxy; and
R₃ and R₄ are, independently of each other, H, halogen, or 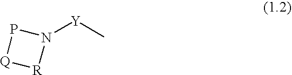
wherein:
P and R are each independently selected from CH₂, CH₂CH₂ and CH₂CHT, wherein T is alkyl;
Q is O, S, NH or NCH₃; and
Y is absent or —(CH₂CH₂O)ₙ—, and n=1 or 2;
with the proviso that at least one of R₃ and R₄ is halogen, and that when R₄ is halogen, R₃ is hydrogen and Y is absent, neither R₁ nor R₂ are alkyl;
or pharmaceutically acceptable salts thereof.

2. The compound according to claim 1, wherein R₁ is aryl unsubstituted or substituted by one to three hydroxy and R₂ is hydrogen; or pharmaceutically acceptable salts thereof.

3. The compound according to claim 1, wherein R₄ is a halogen; or pharmaceutically acceptable salts thereof.

4. The compound according to claim 1, wherein R₃ is a halogen, and R₄ is hydrogen or partial formula (1.2), wherein Q is O, S, NH or NCH₃; or pharmaceutically acceptable salts thereof

5. The compound according to claim 1, wherein R₃ or R₄ is an amine-containing heterocycle, wherein the amine is a ring member of the heterocycle; or pharmaceutically acceptable salts thereof.

6. The compound according to claim 1, wherein R₃ or R₄ is N-methylpiperazine, wherein the N-methylpiperazine is attached through its unsubstituted ring nitrogen; or pharmaceutically acceptable salts thereof.

7. The compound according to claim 1, wherein X is trans CHCH; or pharmaceutically acceptable salts thereof.

8. The compound according to claim 1, wherein R₁ is hydroxyphenyl, 2-hydroxyethanol, or 4-hydroxyphenyl, wherein the point of attachment of 2-hydroxyethanol is at the 1 position and the point of attachment of 4-hydroxyphenyl is at the 1 position; or pharmaceutically acceptable salts thereof.

9. The compound according to claim 1, wherein R₂ is phenyl, substituted phenyl, substituted pyridinyl, thiophenyl, substituted thiophenyl, furanyl, substituted furanyl, thiazole, oxazole or substituted or unsubstituted imidazole; or pharmaceutically acceptable salts thereof.

10. The compound according to claim 1, wherein R₁ is hydrogen and R₂ is not an aromatic ring substituted with a carboxylic acid.

11. The compound according to claim 1, wherein R₁ is hydrogen and R₂ is not an aromatic ring substituted with a carboxylic acid at position 3.

12. The compound according to claim 1, wherein R₂ is an aromatic ring and R₁ is not an acetyl.

13. The compound according to claim 8, wherein R₂ is N-alkyl imidazole, wherein the point of attachment of N-alkyl imidazole is at the 2, 4 or 5 position; or pharmaceutically acceptable salts thereof.

14. The compound according to claim 13, wherein the point of attachment of N-alkyl imidazole is at the 2 position; or pharmaceutically acceptable salts thereof.

15. The compound according to claim 1 which is 7-fluoro-2-[2-(5-nitro-2-furyl)vinyl]-4-(3-aminopyridyl)quinazoline, wherein vinyl is trans; or pharmaceutically acceptable salts thereof.

16. The compound according to claim 1 which is 6-fluoro-2-[2-(5-nitro-2-furyl)vinyl]-4-(p-hydroxyanilino)quinazoline, wherein vinyl is trans; or pharmaceutically acceptable salts thereof.

17. The compound according to claim 1 which is 7-(4-methylpiperazino)-6-fluoro-2-[2-(5-nitro-2-furyl)vinyl]-4-(p-hydroxyanilino)-quinazoline, wherein vinyl is trans; or pharmaceutically acceptable salts thereof.

18. The compound according to claim 1 which is 7-fluoro-2-[2-(5-nitro-2-furyl)vinyl]-4-(p-hydroxyanilino)quinazoline, wherein vinyl is trans; or pharmaceutically acceptable salts thereof.

19. The compound according to claim 1 which is 6-(morpholino)-7-fluoro-2-[2-(5-nitro-2-furyl)vinyl]-4-(p-hydroxyanilino)-quinazoline, wherein vinyl is trans; or pharmaceutically acceptable salts thereof.

20. A method for preventing or treating a bacterial infection in a human or an animal, comprising administering to said human or said animal a prophylactically or therapeutically effective amount of the compound according to claim 1 or pharmaceutically acceptable salts thereof, effective in preventing or treating the bacterial infection.

21. A method for antisepsis of, or disinfecting or sterilizing the surface of an object, including a human, of bacteria, comprising: contacting the object with the compound according to claim 1 or pharmaceutically acceptable salts thereof in an amount and for a time sufficient to achieve a desired degree of antisepsis, disinfection or sterilization.

22. A process for the preparation of the compound according to claim 1, comprising:
a) reacting a compound of formula (1.3) 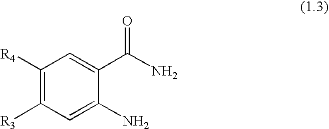
with hydrochloric acid, acetic anhydride and aqueous ammonia, to form a compound of formula (1.4) 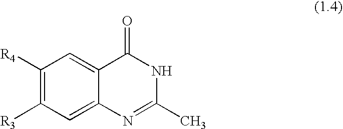
b) reacting the compound of formula 1.4 with 5-nitro-2-furancarboxaldehyde, to form a compound of formula (1.5) 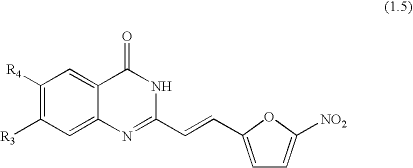
c) reacting the compound of formula 1.5 with phosphorus pentachloride and phosphorus oxychloride to form a compound of formula (1.6) 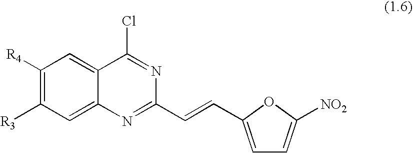
and
d) reacting the compound of formula 1.6 with a compound of the formula (1.7) 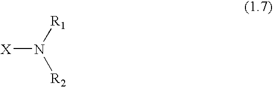
wherein X is H and R₁, R₂, R₃, and R₄ are as defined in claim 1, with the proviso that at least one of R₃ and R₄ is halogen, and that when R₄ is halogen, Y is absent and R₃ is hydrogen, neither R₁ nor R₂ are alkyl.

23. The compound according to claim 1, wherein Y is absent; or pharmaceutically acceptable salts thereof.
